(19) United States
(12) Patent Application Publication
(10) Pub. No.: US 2014/0044784 A1
(43) Pub. Date: Feb. 13, 2014

(54) COMBINED FORMULATION WITH IMPROVED STABILITY

(71) Applicants: Je Hak Kim, Gyeonggi-do (KR); Kyung Wan Nam, Gyeonggi-do (KR); Ju Won Park, Gyeonggi-do (KR)

(72) Inventors: Je Hak Kim, Gyeonggi-do (KR); Kyung Wan Nam, Gyeonggi-do (KR); Ju Won Park, Gyeonggi-do (KR)

(73) Assignee: BORYUNG PHARMACEUTICAL CO., LTD, Seoul (KR)

(21) Appl. No.: 14/004,755

(22) PCT Filed: Mar. 14, 2012
(86) PCT No.: PCT/KR2012/001828

§ 371 Date: Nov. 1, 2013

(30) Foreign Application Priority Data
Mar. 15, 2011 (KR) 10-2011-0023060

(51) Int. Cl. A61K 9/51; A61K 31/505; A61K 31/616

(52) U.S. Cl. CPC A61K 9/51; A61K 31/616; A61K 31/505
USPC 424/474; 514/165; 424/490

(57) ABSTRACT

Disclosed is a combined formulation for oral administration to treat cardiovascular disease, including (a) cholesterol lowering agent mini-tablet having a diameter of 7.5 mm or less, which contain a cholesterol lowering agent, a stabilizer thereof and a pharmaceutically acceptable excipient and have a coating layer on the surface thereof, and (b) antithrombotic agent mini-tablets or mini-pellets having a diameter of 7.5 mm or less, which contain an antithrombotic agent and a pharmaceutically acceptable excipient and include an enteric coating film on the surface thereof. This formulation can improve treatment compliance depending on a combination prescription, and is controlled so that the cholesterol lowering agent is released in the gastrointestinal tracts and the antithrombotic agent is released in the intestines, thus suppressing the reactions and the side-effects between the drugs, inducing synergic effects of these drugs in vivo, and achieving improved stability.

### TECHNICAL FIELD

The present invention relates to a combined formulation for oral administration that can be used in order to treat cardiovascular disease.

### BACKGROUND ART

Aspirin (acetylsalicylic acid) is known to prevent stroke or myocardial infarction due to thrombosis when administered in a small dose for a long period of time to patients at a high risk of disease associated with cardiovascular disease. To this end, 100 mg aspirin in the Tablet form or sustained release capsule form is orally administered once per day. Also heart attack, stroke, and cardiovascular mortalities are known to be reduced by at least 25% via daily administration of a low dose (about 80 mg) of aspirin. The preventive effect of aspirin on cardiovascular disease is based on a variety of mechanisms, of which the inhibition of thrombus is the most critical pharmacological mechanism.

Aspirin irreversibly acetylates and inactivates cyclooxygenase. Cyclooxygenase is essential for synthesizing such materials as prostaglandin, thromboxane A2 and prostacyclin. Prostaglandin is a pro-inflammatory substance, and thromboxane A2 is synthesized in the platelets to cause platelet aggregation, and induces thrombosis.

Also, prostacyclin shows platelet aggregation inhibitory activity. Cyclooxygenase is produced not in the platelets but in the endothelial cells. A low dose of aspirin selectively suppresses cyclooxygenase in the platelets while enabling cyclooxygenase and prostacyclin to be continuously synthesized in the endothelial cells. That is, the main pharmacological effects of aspirin include inflammation inhibition, reduction of platelet aggregation, and attenuation of thrombosis in the blood vessels.

Fats absorbed into the blood, namely, neutral fats, cholesterol, phospholipids, free fatty acids, etc., are combined with proteins and thus provided as a lipoprotein in the form of being dissolved in water, and are the so-called serum lipids. The case where the amount of such lipids in the serum is higher than the normal range is referred to as hyperlipidemia. In order for lipids such as cholesterol to circulate in the blood, because lipids cannot be dissolved in water, they are circulated in the blood in a state of being encapsulated with proteins. This complex of lipids and proteins is called a lipoprotein, and examples of the lipoprotein that transports the cholesterol that is attached thereto include high density lipoprotein (HDL) and low density lipoprotein (LDL). As such, HDL removes cholesterol from the tissue ultimately lowering the risk of arteriosclerosis, whereas LDL functions to accumulate cholesterol on the blood walls thereby increasing the risk of arteriosclerosis.

Hyperlipidemia causes changes in solidification of the blood including platelet aggregation hyperactivity, reduction of platelet solidification time, and a decrease in fibrinolytic system behavior, etc., thus increasing the viscosity of the blood, ultimately incurring a pathological change in the properties and status of the blood and peripheral circulatory disturbance due to vasculitis. Also, atherosclerosis to the artery occurs which makes thrombus which then block the blood vessels. When this phenomenon occurs in the brain, a cerebral infarction ensues, and when this occurs in the coronary artery of the heart, the result is a myocardial infarction, directly resulting in death. Hyperlipidemia is the principal of disease such as angina pectoris, myocardial infarction, stroke, fatty liver and pancreatitis, and particularly is very closely related to the occurrence of arteriosclerosis. In the case where cholesterol is high, the generation of arteriosclerosis may be promoted, and as well, arteriosclerosis is made unstable and thus rapidly progresses into acute myocardial infarction.

A variety of methods are applied to the treatment of hyperlipidemia. Of these, a cholesterol synthesis inhibitor, that is, a 3-hydroxy-3-methylglutaryl-coenzyme A (HMG-CoA) reductase inhibitor, is reputed to be the most effective therapeutic agent for hyperlipidemia.

The pharmacological action of the HMG-CoA reductase inhibitor inhibits the HMG-CoA reductase that takes part in determining the cholesterol synthesis rate in the liver cells, thus decreasing the cholesterol required to synthesize bile acid or the like. Accordingly to supplement it, the number of LDL receptors that cause atherosclerosis is increased, so that many LDLs are removed from the blood, thus decreasing the LDL concentration in the blood thereby exerting efficacy.

Accordingly, the following methods, which include administering both an antithrombotic agent and a therapeutic agent for hyperlipidemia, are known to be useful at treating cardiovascular disease.

For example in U.S. Pat. No. 5,622,985, when a cholesterol lowering agent, in particular pravastatin is used alone or together with an ACE (Angiotensin Converting Enzyme) inhibitor, the risk of a second heart attack may be decreased in patients who have substantially normal cholesterol level. However, this patent is problematic because two agents should be administered together.

Also, U.S. Pat. No. 5,140,012 discloses the use of pravastatin alone or in combination with an ACE inhibitor in order to prevent the risk of restenosis following angioplasty, but there is the inconvenience of administration and no research has been carried out into preventing or treating another cardiovascular disease other than the above, thus revealing the limits of application of the related studies.

Also, EP 457,514 discloses the use of a concept similar to U.S. Pat. Nos. 5,622,985 and 5,140,012. This patent is merely an extension of the above two patents, and no research has been conducted into mixing aspirin with another cholesterol lowering agent to introduce or expand a novel concept and thus the range of research has been limited.

Also, U.S. Pat. No. 6,248,729 discloses a combination of an ADP receptor-blocking platelet inhibitor and an angiotensin receptor and the use thereof, and a combination of an ADP receptor-blocking platelet inhibitor and an ACE inhibitor and the use thereof. However, the combination of a cholesterol lowering agent and aspirin and the use thereof are not mentioned therein.

On the other hand, Korean Patent Laid-Open No. 10-2006-0091762 discloses combined pellets containing HMG-CoA reductase inhibitor and enteric coated aspirin for preventing arteriosclerosis in hyperlipidemia patients. Specifically, the above patent provides a dosage form having a multilayer structure comprising a main layer comprising inert sugar particles coated with aspirin, an enteric coating layer on the outer surface of the main layer, and an outer layer comprising HMG-CoA reductase inhibitor applied onto the outer surface of the enteric coating layer. However, mass production of such a dosage form having a multilayer structure is very difficult. For example, expensive specific equipment is essentially required, and also coating should be carried out at high temperature for a long period of time, adversely affecting the stability of the drug. The production yield may be lower than when the respective drugs are individually coated. Particularly in the course of coating, mutual physical reactions based on the direct contact between the HMG-CoA reductase inhibitor and aspirin may directly cause the decrease of the dissolution rates, thereby drastically deteriorating stability. Aspirin which is an acidic drug may react with a basic compound or basic ester to thus cause hydrolysis of the aspirin or the decomposition of other compounds, and may react with a compound unstable to an acid, such as pravastatin, thus decomposing it.

U.S. Pat. No. 6,235,311 discloses a bilayered tablet wherein a buffer layer is interposed between a first layer containing aspirin and a second layer containing a statin-based drug, in order to minimize the interactions of aspirin and the statin drugs.

However, the above patent is problematic because expensive production equipment is additionally required to produce the bilayered tablet, and even when the bilayered tablet is designed, limitations are imposed on how the abnormal reactions may be fundamentally blocked because of the contact between the drugs.

Korean Patent Laid-Open No. 10-2008-0052011 discloses a combined formulation comprising first pellets including an antithrombotic agent core layer and an enteric coating layer and second pellets including an inert particle core layer, a cholesterol lowering agent intermediate layer and an outer coating layer. However, a cholesterol lowering drug such as rosuvastatin or pravastatin that is unstable to an acid requires the use of a stabilizer to form the intermediate layer of the second pellets. In the case of the above formulation, it is difficult to prepare the intermediate layer comprising a mixture of the drug and the stabilizer, and the uniformity of the prepared intermediate layer is not ensured, and the storage stability of the cholesterol lowering agent weak to an acid still remains a problem.

### PRIOR ART REFERENCE

### Patent Literature

Korean Patent Laid-Open No. 10-2009-0030452
Korean Patent No. 10-0870396

### Non-Patent Literature

C. M. Lopes et al., Int. J. Pharm. 323(1-2), 93-100 (2006)
T. Riis et al., Eur. J. Pharm. Biopharm. 65(1). 78-84 (2007)
C. M. Lopes et al., Drug. Dev. Ind. Pharm. 32(1) 95-106. (2006)
I. Tomuta and S. E. Leucuta. Drug. Dev. Ind. Pharm. 33(1) 1070-1077 (2007)
A. Spadoni, Pharm. Proc. Published online (Jan. 2001), www.thefreelibrary.com/Evaluation+Of+An+Alternative+Solid+Dosage+Form+To+Entric+Coated . . . −a074218471, accessed Oct. 22, 2010.
M. Ishida et al., Int. J. Pharm, 359(1-2), 46-52 (2008)
L. Ho et al., J. Control. Rel. 127(1), 79-87 (2008).

### DISCLOSURE OF INVENTION

### Technical Problem

Accordingly, the present invention has been made keeping in mind the above problems occurring in the related art, and an object of the present invention is to provide a combined formulation for oral administration for the treatment of cardiovascular disease, which contains an antithrombotic agent and a cholesterol lowering agent, thus improving or preventing unstable drug reactions or hydrolysis due to the interactions between the two drugs, thereby maximizing therapeutic effects and improving storage stability.

### Solution to Problem

In order to accomplish the above objects, the present invention provides a combined formulation for oral administration that is for treating cardiovascular disease, comprising (a) a cholesterol lowering agent mini-tablet having a diameter of 7.5 mm or less, which contains a cholesterol lowering agent, a stabilizer and a pharmaceutically acceptable excipient and has a coating layer on the surface thereof, and (b) an antithrombotic agent mini-tablet or mini-pellet having a diameter of 7.5 mm or less, which contains an antithrombotic agent and a pharmaceutically acceptable excipient and includes an enteric coating film on the surface thereof.

The combined formulation for oral administration to treat cardiovascular disease according to the present invention can improve or prevent unstable drug reactions or hydrolysis due to interactions between the antithrombotic agent and the cholesterol lowering agent to thus maximize therapeutic effects. In particular, the present invention provides a combined formulation in a single dosage form whose storage stability of an antithrombotic agent such as a salicylic acid derivative and a cholesterol lowering agent unstable to an acid, for example, rosuvastatin, atorvastatin, pitavastatin and pravastatin has been optimized.

### Advantageous Effects of Invention

According to the present invention, a combined oral administration formulation for treating cardiovascular disease improves upon the conventional inconvenience of an antithrombotic agent and a cholesterol lowering agent having to be taken separately. The formulation of the present invention is configured such that, upon administration, the cholesterol lowering agent is first dissolved to thus reduce the amount of lipid such as cholesterol which is present in an excessive amount in the blood, and aspirin coated with an enteric coating material is dissolved when reaching the upper portion of the small intestine, thus inhibiting platelet aggregation to thereby prevent the formation of thrombus, so that the dissolution and bioavailability of two such drugs can be prevented from being decreased in vivo by the direct contact of the drugs. Particularly, although the storage stability of the conventional combined formulations comprising the antithrombotic agent, which causes the environment to be acidic, and the cholesterol lowering agent, which is weak to acid, is problematic, the dosage form of the present invention has improved upon storage stability of a combined formulation of two drugs.

### BRIEF DESCRIPTION OF DRAWINGS

FIG. 1 schematically shows a capsule dosage form filled with cholesterol lowering agent mini-tablets and antithrombotic agent mini-tablets or mini-pellets.

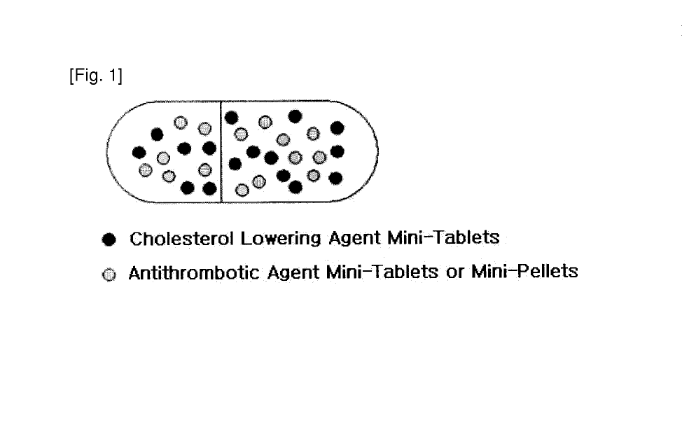

### MODE FOR THE INVENTION

Hereinafter, a detailed description will be given of the present invention.

(1) Cholesterol Lowering Agent Mini-Tablets

According to the present invention, cholesterol lowering agent mini-tablets include a cholesterol lowering agent as a pharmacologically active ingredient, and additionally its stabilizer and a pharmaceutically acceptable excipient, and have a coating layer on the surface thereof, with a diameter of 7.5 mm or less.

The cholesterol lowering agent of the present invention to be optimized storage stability is ones that are unstable in an acidic environment, and typical examples thereof include rosuvastatin, rosuvastatin calcium, atorvastatin, atorvastatin calcium, pitavastatin, pravastatin, and pharmaceutically acceptable salts thereof. These drugs include solvate (including hydrate), crystalline and amorphous forms, which may be used alone or in mixtures of two or more. Because the above drugs are unstable in an acidic environment, an alkalizing agent must be used as the stabilizer.

The stabilizer used in the cholesterol lowering agent mini-tablets of the present invention, which functions to stabilize a drug unstable to an acid, may include a pharmaceutically acceptable alkalizing agent that is commonly used in the pharmaceutical industry. Such an alkalizing agent may include butylated hydroxytoluene (BHT), dibutylhydroxytoluene (DHT), butylated hydroxyanisole (BHA), sodium sulfite, sodium pyrosulfite, sodium bisulfite, propyl gallate, calcium phosphate, etc. Of these, calcium phosphate is preferably used.

The amount of the stabilizer may be 0.00001˜5 and preferably 0.00002˜3 parts by weight based on 1 part by weight of the cholesterol lowering agent which is the pharmacologically active ingredient. If the amount of the stabilizer is less than 0.00001 parts by weight based on 1 part by weight of the active ingredient, drug stabilization effects cannot be expected. In contrast, if the amount thereof is greater than the above upper limit, it may exceed the daily acceptable amount of the stabilizer, so that its safety may undesirably be problematic.

The cholesterol lowering agent mini-tablets of the present invention may further include an excipient, a carrier and other additives, which are pharmaceutically acceptable, in addition to the cholesterol lowering agent and the stabilizer. Such an excipient, carrier and other additives may include a diluent, a binder, a lubricant, a disintegrant, etc. that are commonly used in the pharmaceutical industry.

The diluent may include any diluent commonly used in the pharmaceutical industry, and for example, lactose, microcrystalline cellulose, starch, mannitol, etc., are typical, and in addition, white sugar, sorbitol, and inorganic salts such as dibasic calcium phosphate, tribasic calcium phosphate, aluminum silicate, calcium sulfate, etc. may be used, but the present invention is not limited thereto. Particularly useful is microcrystalline cellulose. The amount of the diluent may be appropriately determined by those skilled in the art, and for example may be properly set in the range of 0.0001˜200 parts by weight based on 1 part by weight of the active ingredient.

The binder may include any binder commonly used in the pharmaceutical industry, and for example includes but is not limited to starch, microcrystalline cellulose, highly dispersible silica, mannitol, sucrose, lactose, polyethylene glycol, polyvinylpyrrolidone, hydroxypropylmethylcellulose, hydroxypropylcellulose, carboxymethylcellulose sodium, pregelatinized starch, natural gum, synthetic gum, polyvinylpyrrolidone copolymers, povidone, copovidone, gelatin or mixtures thereof. Particularly useful is anhydrous lactose, povidone, copovidone, or hydroxypropylcellulose. The amount of the binder may be appropriately determined by those skilled in the art, and for example may be properly set in the range of 0.0001˜200 parts by weight based on 1 part by weight of the active ingredient.

The lubricant may include any lubricant commonly used in the pharmaceutical industry, and for example includes but is not limited to talc, stearic acid, magnesium stearate, calcium stearate, sodium lauryl sulfate, hydrogenated vegetable oil, sodium benzoate, sodium stearyl fumarate, glyceryl behenate, glyceryl monolaurate, glyceryl monostearate, glyceryl palmitostearate, polyethyleneglycols or mixtures thereof, magnesium laurylsulfate, sodium benzoate, polyoxyethylene monostearate, glyceryltriacetate, sucrose monolaurate, zinc stearate, hardened vegetable oil, light liquid paraffin, paraffins, and wax. Particularly useful is magnesium stearate. The amount of the lubricant may be appropriately determined by those skilled in the art, and for example may be properly set in the range of 0.0001˜100 parts by weight based on 1 part by weight of the active ingredient.

The disintegrant may include any disintegrant commonly used in the pharmaceutical industry, and for example includes but is not limited to starch or a modified starch, such as sodium starch glycolate, corn starch, potato starch or pregelatinized starch; clay such as bentonite, montmorillonite, or veegum; cellulose such as microcrystalline cellulose, hydroxypropylcellulose or carboxymethylcellulose; algins such as sodium alginate or alginic acid; crosslinked cellulose such as croscarmellose sodium or the like; gums such as guar gum, xanthan gum or the like; crosslinked polymer such as crosslinked polyvinylpyrrolidone (crospovidone) or the like; effervescent agents, such as sodium bicarbonate, citric acid or the like, or mixtures thereof. Particularly useful is crospovidone. The amount of the disintegrant may be appropriately determined by those skilled in the art, and for example may be properly set in the range of 0.0001˜200 parts by weight based on 1 part by weight of the active ingredient.

In the present invention, the mini-tablets include a pharmaceutically active ingredient and an optionally appropriate excipient, and is provided in the form of a pharmaceutically orally administrable solid obtained by a process including compression molding, with a diameter (which means the largest longitudinal dimension of the tablet) of 7.5 mm or less, 5 mm or less, 4.5 mm or less, 4.0 mm or less, 3.5 mm or less, 3.0 mm or less, 2.5 mm or less, or 1.0˜7.5 mm, 1.0˜5 mm, 1.0˜4.5 mm, 1.0˜4.0 mm, 1.0˜3.5 mm, 1.0˜3.0 mm, 1.0˜2.5 mm, 1.5˜7.5 mm, 1.5˜5 mm, 1.5˜4.5 mm, 1.5˜4.0 mm, 1.5˜3.5 mm, 1.5˜3.0 mm, 1.5˜2.5 mm. Preferably the diameter of the tablets is 1.5˜7.5 mm, and more preferably 1.5˜2.5 mm. The shape of the mini-tablets may be an arbitrary shape that is convenient for experts, and for example, may be either spherical or cylindrical. In one embodiment of the present invention, the mini-tablets have a convex shape while being roundish.

The mini-tablets of the present invention may be prepared by a mini-tablet preparation process which is commonly used in the art, for example, wet granule compression, dry granule compression, direct compression, etc. Preferably, mini-tablets may be directly prepared by mixing respective ingredients and compressing the mixture using a tablet press equipped with multi-tip punches. Alternatively, respective ingredients may be mixed, granulated, dried, milled and compressed into tablets in one or more steps, thus obtaining the mini-tablets. In one embodiment, a wet granulation process which is widely known in the art may be applied. For example, a pharmacologically active ingredient, a filler, a polymer and a sufficient amount of granulation fluid, for example, water are mixed, granulated, dried and milled thus forming granules. The dried granules are milled to an appropriate particle size of D50 (median particle size) of for example, 50˜300 μm, 100˜300 μm, or 100˜200 μm, followed by mixing the granules with the other ingredients at high shear rate and compressing the mixture into mini-tablets using a tablet press equipped with multi-tip punches. The mini-tablets of the present invention may be prepared via compression at a pressure of 0.5˜1.5 KN. If the pressure falling outside the above range is applied, it is difficult to prepare such mini-tablets.

The cholesterol lowering agent mini-tablets of the present invention have an outer coating layer.

The outer coating layer may be formed using a known material that can be used in the pharmaceutical industry to coat a cholesterol lowering agent. The outer coating layer may be formed using a coating layer forming material comprising a polymer, a plasticizer, an anti-adhesive agent, a pigment and a light-shielding agent, and other materials such as a fragrant or a sweetener may be additionally added, as necessary.

The polymer may be one or more selected from the group consisting of cellulose ethers {e.g. hydroxypropylmethylcellulose (Hypromellose), hydroxypropylcellulose, methylcellulose, ethylcellulose, etc.}, vinyl polymers {e.g. polyvinyl alcohol, polyvinyl acetate phthalate, polyvinyl pyrrolidone, etc.}, and acrylic polymers {e.g. methacrylic acid co-polymers, etc.}.

The plasticizer may include a water-soluble plasticizer such as polyethylene glycols, glycerol, propyleneglycol, phthalate esters, triacetin, acetylated monoglyceride, citrate esters, etc., and a water-insoluble plasticizer such as lecithin, etc.

The anti-adhesive agent may include any anti-adhesive agent which is pharmaceutically acceptable, for example, talc, etc.

The pigment and the light-shielding agent may be one or more selected from the group consisting of water-soluble dyes, synthetic pigments {e.g. aluminum lakes, titanium dioxide, iron oxides, talc, calcium sulfate, calcium carbonate, magnesium carbonate, etc.} and natural pigments {e.g. riboflavin, carotenoid, anthocyanin, carmine, curcumin, chlorophyll, etc.}.

The other materials may include a fragrant such as vanillin, a sweetener, etc.

The above coating layer forming materials may be mixed with a solvent such as purified water, ethanol (fermented alcohol), methyl chloride (MC), isopropanol (IPA), etc., which are typically used in the pharmaceutical industry, thus preparing a coating solution.

Preferably useful as the coating layer is Opadry (Colorcon; 415 Moyer Blvd., P.O. Box 4, West Point, Pa. 19486-0024, USA).

Also, any anti-moisture barrier coating which is commonly used may be applied, and for example, a coating substrate suitable for use in the anti-moisture barrier coating, such as Opadry AMB (Anti-Moisture Barrier) alone, Opadry AMB and Eudragit combinations, Opadry Acryl-EZE, etc., may be used.

The coating layer forming material may be used in an amount of 0.01˜5.0 parts by weight, and preferably 0.05˜2.5 parts by weight based on 1 part by weight of the cholesterol lowering agent.

The outer coating may be performed using a coating process typically used in the pharmaceutical industry.

The mini-tablets of the present invention preferably have a weight of 2˜50 mg.

(2) Antithrombotic agent mini-tablets or mini-pellets

According to the present invention, antithrombotic agent mini-tablets or mini-pellets include an antithrombotic agent as a pharmacologically active ingredient, and have an enteric coating layer on the surface thereof, with a diameter of 7.5 mm or less.

The combined formulation of the present invention is a dosage form adapted to maintain the stability of a cholesterol lowering agent which is weak to an acid and an acidic antithrombotic agent, and thus, the antithrombotic agent used in the present invention is an acidic drug. Such a drug may include a salicylic acid derivative. The salicylic acid derivative may be one or more selected from the group consisting of sodium salicylate, magnesium salicylate (including tetrahydrate), salicylsalicylic acid (salsalate) and aspirin (acetylsalicylic acid). Also the drug includes solvate (including a hydrate), crystalline and amorphous forms. Particularly useful in the present invention is aspirin.

The antithrombotic agent mini-tablets or mini-pellets according to the present invention may include an excipient, a carrier and other additives, which are pharmaceutically acceptable, in addition to the above antithrombotic agent. Such an excipient, carrier and other additives may include a diluent, a binder, a lubricant, a disintegrant, etc., which are typically used in the pharmaceutical industry, and specific examples thereof may include those that were mentioned above.

In the antithrombotic agent mini-tablets, the contents regarding the preparation, diameter and so on of the cholesterol lowering agent mini-tablets as mentioned above may be applied with appropriate modifications. In the antithrombotic agent mini-tablets of the present invention, the diameter may be 7.5 mm or less, 5 mm or less, 4.5 mm or less, 4.0 mm or less, 3.5 mm or less, 3.0 mm or less, 2.5 mm or less, or 1.0˜7.5 mm, 1.0˜5 mm, 1.0˜4.5 mm, 1.0˜4.0 mm, 1.0˜3.5 mm, 1.0˜3.0 mm, 1.0˜2.5 mm, 1.5˜7.5 mm, 1.5˜5 mm, 1.5˜4.5 mm, 1.5˜4.0 mm, 1.5˜3.5 mm, 1.5˜3.0 mm, 1.5˜2.5 mm. Preferably the diameter is 1.5˜7.5 mm, and more preferably 1.5˜2.5 mm.

In the present invention, the mini-pellets include a pharmaceutically active ingredient and an optionally appropriate excipient, and are an agglomeration of spherical particles formulated via process including extrusion and spheronization, and the diameter (the largest longitudinal dimension of the pellet) thereof is 7.5 mm or less, 5 mm or less, or 4.5 mm or less, 4.0 mm or less, 3.5 mm or less, 3.0 mm or less, 2.5 mm or less, 2.0 mm or less, or 0.5˜7.5 mm, 0.5˜5 mm, 0.5˜4.5 mm, 0.5˜4.0 mm, 0.5˜3.5 mm, 0.5˜3.0 mm, 0.5˜2.5 mm, 0.5˜2.0 mm. Preferably the diameter is 0.5˜7.5 mm, and more preferably 0.5˜2.0 mm.

The antithrombotic agent mini-pellets may be prepared via a pellet forming process commonly used in the art, for example, an extrusion-spheronization technique, etc.

The antithrombotic agent mini-tablets or mini-pellets according to the present invention have an enteric coating film.

The enteric coating means a coating that delays the release of a drug from mini-tablets or mini-pellets until the drug reaches the duodenum, the ileum and/or the cecum/colon and is released thereto. Most enteric coatings are known to be pH-sensitive in the art, but the enteric coating used in the present invention includes both a pH-sensitive coating and a pH-independent coating.

In the present invention, the enteric coating film of the antithrombotic agent mini-tablets or mini-pellets may contain an excipient, a binder and a disintegrant. The coating substrate used in the enteric coating film may include any one useful in the pharmaceutical industry, for example ethylcellulose, cellulose acetate, polyvinyl acetate, cellulose butyrate phthalate, cellulose hydrogen phthalate, cellulose propionate phthalate, polyvinylacetate phthalate, cellulose acetate phthalate (CAP), cellulose acetate trimellitate, hydroxypropyl methylcellulose phthalate (HPMCP), methacrylic acid (Eudragit), polyvinylacetate, hydroxypropyl methylacetate, dioxypropylmethylcellulose succinate, carboxymethylethyl cellulose, hydroxypropylmethylcellulose acetate succinate and mixtures thereof, and copolymers formed from polymers thereof and acrylic acid, methacrylic acid or esters thereof.

More specifically, a pH dependent polymer, for example, methacrylic acid and methacrylic acid ester copolymer, for example, methacrylic acid copolymer, for example, a Eudragit product that is soluble above pH 5.5 (for example, Eudragit L30D55) may be included. Other Eudragit products may include Eudragit L100-55 (soluble over pH 5.5), Eudragit L100 (soluble over pH 6.0) and Eudragit S100 (soluble over pH 7.0).

The enteric coating film may further include a plasticizer. During the film coating process, a plasticizer that aids the formation of the film, for example, acetyltriethyl citrate or triethyl citrate, for example, triethyl citrate (Citroflex) may be additionally included.

Also, the enteric coating film may further include a lubricant. The pharmaceutical composition of the present invention may further include a lubricant that prevents adhesion during the film coating process, for example, talc, kaolin, or glycerol monostearate, for example glycerol monostearate (Imwitor 900K).

Also, the enteric coating film may further include a surfactant. The pharmaceutical composition of the present invention may further include a surfactant that enables the formation of a uniform film mixture, for example, sodium laurylsulfate, polyethyleneglycol or polysorbate, for example, polysorbate 80 (Crillet 4HP).

The amount of the enteric coating film forming material may be 0.01˜0.7 parts by weight, and preferably 0.05˜0.5 parts by weight based on 1 part by weight of aspirin.

The enteric coating film may be formed by spraying the above enteric coating film forming material onto the mini-tablets or mini-pellets.

The mini-tablets or mini-pellets of the present invention preferably have a weight of 0.5˜30 mg.

(3) Combined Formulation

The combined formulation of the present invention may be produced by separately preparing the cholesterol lowering agent mini-tablets and the antithrombotic agent mini-tablets or mini-pellets as mentioned above and filling hard capsules with them together.

In the present invention, the formulation may be a hard or soft capsule.

The capsules may be hard gelatin or hydroxymethylcellulose (HPMC) capsules. In one embodiment of the present invention, the capsules may contain a particulate filler, for example, microcrystalline cellulose. Each capsule according to the present invention may be filled with 1˜50 cholesterol lowering agent mini-tablets and 1˜50 antithrombotic agent mini-tablets or mini-pellets; or 2˜30 cholesterol lowering agent mini-tablets and 2˜30 antithrombotic agent mini-tablets or mini-pellets; or 3˜20 cholesterol lowering agent mini-tablets and 3˜20 antithrombotic agent mini-tablets or mini-pellets, but the present invention is not limited thereto.

The amount of the antithrombotic agent may be 0.5˜500 mg and preferably 30˜300 mg per capsule. Specifically, the amount of aspirin is preferably 75˜120 mg per capsule.

The amount of the cholesterol lowering agent may be 1˜300 mg, preferably 2˜100 mg and more preferably 3˜50 mg per capsule.

A single dose of the formulation may vary depending on the severity of disease, age, gender, complication, etc. of a patient.

The combined formulation of the present invention may be used to treat or prevent cardiovascular disease, and the kinds of cardiovascular disease are well known in the art, and typical examples thereof include but are not limited to hypertension, stroke, angina pectoris, hyperlipidemia, myocardial infarction, arteriosclerosis, etc.

The following examples, test examples and comparative examples which are set forth to illustrate but are not to be construed as limiting the scope of the present invention may provide a better understanding of the present invention.

### PREPARATION EXAMPLE

In order to prepare a dosage form according to the present invention, antithrombotic agent mini-tablets or mini-pellets for a capsule, and cholesterol lowering agent mini-tablets for a capsule were prepared as follows.

### Preparation Example A-1

### Preparation of Aspirin Mini-Tablets

Ingredients and amounts of aspirin mini-tablets for a pharmaceutical dosage form according to the present invention are shown in the Table 1 below.

TABLE 1
Composition of Aspirin Mini-tablets

| | Tablet Part | Coating part |
|---|---|---|
| | (unit: | (unit: |
| Ingredients | mg/capsule) | mg/capsule) |
| Aspirin | 100 | |
| Microcrystalline Cellulose | 30 | |
| Kollidon VA 64(Binder) | 10 | |
| Hydroxypropyl Methylcellulose | | 8.6 |
| Phthalate | | |
| Diethyl Phthalate | | 0.74 |
| Wheat Starch | | 0.27 |
| Magnesium Stearate | | 0.27 |
| Ethanol | | 48 |
| Methylene Chloride | | 90 |
| Total | 140 | 147.88 |

(1) Preparation of Mini-Tablets

Aspirin, microcrystalline cellulose, and Kollidon VA 64 (available from BASF) were mixed in the amounts shown in the Table 1 using a V-mixer, after which the resulting mixture was placed in a tablet press equipped with multi-tip punches (KT-10S, available from Sejong Pharmatech) and compressed at a pressure of 1 KN, thus preparing circular mini-tablets having a diameter of 1.5˜7.5 mm and a weight of 2˜50 mg.

(2) Enteric Coating

Pharmaceutically acceptable enteric coating materials (hydroxypropyl methylcellulose phthalate, diethyl phthalate, wheat starch and magnesium stearate) were mixed in the amounts shown in the Table 1, dissolved in ethanol and methylene chloride solvents, and then applied onto the above mini-tablets using a fan coater (LabCoat-M, available from OHARA, Canada, inner outlet of the fan: 1.2 Ø) or a flow coater (GPCP-1, available from Glatt, Germany) to coat the above mini-tablets.

### Preparation Example A-2

### Preparation of Aspirin Mini-Tablets

Ingredients and amounts of aspirin mini-tablets for a pharmaceutical dosage form according to the present invention are shown in the Table 2 below.

TABLE 2
Composition of Aspirin Mini-tablets

| | Tablet Part | Coating part |
|---|---|---|
| | (unit: | (unit: |
| Ingredients | mg/capsule) | mg/capsule) |
| Aspirin | 100 | |
| Microcrystalline Cellulose | 6.4 | |
| Hydroxypropylcellulose | 4.4 | |
| (Binder) | | |
| Purified water | 0.3 | |
| Hydroxypropyl Methylcellulose | | 8.6 |
| Phthalate | | |
| Diethyl Phthalate | | 0.74 |
| Wheat Starch | | 0.27 |
| Magnesium Stearate | | 0.27 |
| Ethanol | | 48 |
| Methylene Chloride | | 90 |
| Total | 111.1 | 147.88 |

(1) Preparation of Mini-Tablets

Aspirin and microcrystalline cellulose were mixed in the amounts of the Table 2 using a high speed mixer (available from Sejong Pharmatech), and then granulated with a previously prepared binder solution (a solution of hydroxypropylcellulose dispersed in purified water). The granules thus obtained were dried, sieved to have a predetermined particle size (Mesh Size: No. 25-30 sieve), and mixed using a V-mixer, after which the mixture was placed in a tablet press equipped with multi-tip punches (KT-10S, available from Sejong Pharmatech) and compressed at a pressure of 1 KN, thus preparing circular mini-tablets having a diameter of 1.5˜7.5 mm and a weight of 2˜50 mg.

(2) Enteric Coating

Pharmaceutically acceptable enteric coating materials (hydroxypropyl methylcellulose phthalate, diethyl phthalate, wheat starch and magnesium stearate) were mixed in the amounts shown in the Table 2, dissolved in ethanol and methylene chloride solvents, and then applied onto the above mini-tablets using a fan coater (LabCoat-M, available from OHARA, Canada, inner outlet of the fan: 1.2 Ø) or a flow coater (GPCP-1, available from Glatt, Germany) to coat the above mini-tablets.

### Preparation Example A-3

### Preparation of Aspirin Mini-Pellets

Ingredients and amounts of aspirin mini-pellets for a pharmaceutical dosage form according to the present invention are shown in the Table 3 below.

TABLE 3
Composition of Aspirin Mini-pellets

| | Core layer | Coating part |
|---|---|---|
| | (unit: | (unit: |
| Ingredients | mg/capsule) | mg/capsule) |
| Aspirin | 100 | |
| Microcrystalline Cellulose | 6.4 | |
| Hydroxypropylcellulose | 4.4 | |
| (Binder) | | |
| Purified water | 0.3 | |
| Hydroxypropyl Methylcellulose | | 8.6 |
| Phthalate | | |
| Diethyl Phthalate | | 0.74 |
| Wheat Starch | | 0.27 |
| Magnesium Stearate | | 0.27 |
| Ethanol | | 48 |
| Methylene Chloride | | 90 |
| Total | 111.1 | 147.88 |

(1) Preparation of Aspirin Pellets

Aspirin and microcrystalline cellulose were mixed in the amounts of the Table 3 using a high speed mixer (available from Sejong Pharmatech) and granulated with a previously prepared binder solution (a solution of hydroxypropylcellulose dispersed in purified water). The granules thus obtained were extruded using an extruder (available from Sejong Pharmatech), and spheronized using a Marumerizer (available from Sejong Pharmatech), thus preparing spherical mini-pellets having a diameter of 0.5˜7.5 mm and a weight of 0.5˜50 mg.

(2) Enteric Coating

Pharmaceutically acceptable enteric coating materials (hydroxypropyl methylcellulose phthalate, diethyl phthalate, wheat starch and magnesium stearate) were mixed in the amounts shown in the Table 3, dissolved in ethanol and methylene chloride solvents, and then applied onto the above mini-pellets using a flow coater (GPCP-1, available from Glatt, Germany) to coat the above mini-pellets.

### Preparation Example B-1

### Preparation of Rosuvastatin Mini-Tablets

Ingredients and amounts of rosuvastatin mini-tablets for a pharmaceutical dosage form according to the present invention are shown in the Table 4 below.

TABLE 4
Composition of Rosuvastatin Calcium Mini-tablets

| | Tablet Part | Coating part |
|---|---|---|
| | (unit: | (unit: |
| Ingredients | mg/capsule) | mg/capsule) |
| Rosuvastatin Calcium | 10.4 | |
| Microcrystalline Cellulose | 34.82 | |
| Calcium Phosphate | 10.9 | |
| (Stabilizer) | | |
| Anhydrous Lactose | 94.5 | |
| Crospovidone | 7.5 | |
| Magnesium Stearate | 1.88 | |
| Opadry | | 10.0 |
| Ethanol | | 72.0 |
| Purified water | | 18.0 |
| Total | 160 | 100.0 |

(1) Preparation of Mini-Tablets

The ingredients of the tablet part were mixed in the amounts of the Table 4 using a V-mixer, and this mixture was placed in a tablet press equipped with multi-tip punches (KT-10S, available from Sejong Pharmatech) and compressed at a pressure of 1 KN, thus preparing circular mini-tablets having a diameter of 1.5˜7.5 mm and a weight of 2˜50 mg.

(2) Outer Coating

Opadry was dissolved in ethanol and purified water in the amounts of the Table 4, and then applied onto the above mini-tablets using a fan coater (LabCoat-M, available from OHARA, Canada, inner outlet of the fan: 1.2 Ø) or a flow coater (GPCP-1, available from Glatt, Germany) to coat the above mini-tablets.

### Preparation Example B-2

### Preparation of Rosuvastatin Mini-Tablets

Ingredients and amounts of rosuvastatin mini-tablets for a pharmaceutical dosage form according to the present invention are shown in the Table 5 below.

TABLE 5
Composition of Rosuvastatin Calcium Mini-tablets

| | Tablet Part | Coating part |
|---|---|---|
| | (unit: | (unit: |
| Ingredients | mg/capsule) | mg/capsule) |
| Rosuvastatin Calcium | 10.40 | |
| Microcrystalline Cellulose | 57.00 | |
| Calcium Phosphate (Stabilizer) | 26.00 | |
| Copovidone (Kollidon VA 64, Fine) | 11.00 | |
| Anhydrous Lactose | 18.60 | |
| Crospovidone | 11.00 | |
| Light Anhydrous Silicic Acid | 3.50 | |
| (Aerosil 200VV) | | |
| Magnesium Stearate | 2.50 | |
| Opadry | | 10.0 |
| Ethanol | | 72.0 |
| Purified water | | 18.0 |
| Total | 140.00 | 100.0 |

(1) Preparation of Mini-Tablets

The ingredients of the tablet part were mixed in the amounts of the Table 5 using a V-mixer, and the mixture was placed in a tablet press equipped with multi-tip punches (KT-10S, available from Sejong Pharmatech) and compressed at a pressure of 1 KN, thus preparing circular mini-tablets having a diameter of 1.5˜7.5 mm and a weight of 2˜50 mg.

(2) Outer Coating

Opadry was dissolved in ethanol and purified water in the amounts of the Table 5, and then applied onto the above mini-tablets using a fan coater (LabCoat-M, available from OHARA, Canada, inner outlet of the fan: 1.2 Ø) or a flow coater (GPCP-1, available from Glatt, Germany) to coat the above mini-tablets.

### Preparation Example B-3

### Preparation of Rosuvastatin Mini-Tablets

Ingredients and amounts of rosuvastatin mini-tablets for a pharmaceutical dosage form according to the present invention are shown in the Table 6 below.

TABLE 6
Composition of Rosuvastatin mini-tablets

| | Tablet Part | Coating part |
|---|---|---|
| | (unit: | (unit: |
| Ingredients | mg/capsule) | mg/capsule) |
| Rosuvastatin Calcium | 10.4 | |
| Microcrystalline Cellulose | 57.0 | |
| Calcium Phosphate | 26.0 | |
| (Stabilizer) | | |
| Lactose | 11.0 | |
| Crospovidone | 18.6 | |
| Povidone K-25 (Binder) | 11.0 | |
| Magnesium Stearate | 3.5 | |
| Purified water | 2.5 | |
| Opadry | | 10.0 |
| Ethanol | | 72.0 |
| Purified water | | 18.0 |
| Total | 140 | 100.0 |

(1) Preparation of Mini-Tablets

Rosuvastatin calcium, microcrystalline cellulose and lactose were mixed in the amounts of the Table 6 using a high speed mixer (available from Sejong Pharmatech) and granulated with a previously prepared binder solution (a solution of povidone K-25 dispersed in purified water).

The granules thus obtained were dried, sieved (Mesh Size: No. 25-30 sieve), and then mixed with calcium phosphate, crospovidone, and magnesium stearate using a V-mixer, after which the resulting mixture was placed in a tablet press equipped with multi-tip punches (KT-10S, available from Sejong Pharmatech) and compressed at a pressure of 1 KN, thus preparing circular mini-tablets having a diameter of 1.5˜7.5 mm and a weight of 2˜50 mg.

(2) Outer Coating

Opadry was dissolved in ethanol and purified water in the amounts of the Table 6, and then applied onto the above mini-tablets using a fan coater (LabCoat-M, available from OHARA, Canada, inner outlet of the fan: 1.2 Ø) or a flow coater (GPCP-1, available from Glatt, Germany) to coat the above mini-tablets.

### Preparation Example B-4

### Preparation of Rosuvastatin Mini-Tablets

Three types of coated mini-tablets were respectively prepared using the methods of Preparation Examples B-1, B-2 and B-3, with the exception that, in the Tables 4, 5 and 6 given in Preparation Examples B-1, B-2 and B-3, 5 mg of rosuvastatin was used instead of 10.4 mg of rosuvastatin calcium, and the amounts of the other ingredients of the tablet part were decreased by ½.

### Preparation Example B-5

### Preparation of Rosuvastatin Mini-Tablets

Three types of coated mini-tablets were respectively prepared using the methods of Preparation Examples B-1, B-2 and B-3, with the exception that, in the Tables 4, 5 and 6 given in Preparation Examples B-1, B-2 and B-3, 20 mg of rosuvastatin was used instead of 10.4 mg of rosuvastatin calcium, and the amounts of the other ingredients of the tablet part were doubled.

### Preparation Example B-6

### Preparation of Atorvastatin Calcium Mini-Tablets

Three types of coated mini-tablets were respectively prepared using the methods of Preparation Examples B-1, B-2 and B-3, with the exception that, in the Tables 4, 5 and 6 given in Preparation Examples B-1, B-2 and B-3, 20 mg of atorvastatin calcium was used instead of 10.4 mg of rosuvastatin calcium.

### Preparation Example B-7

### Preparation of Atorvastatin Calcium Mini-Tablets

Three types of coated mini-tablets were respectively prepared using the methods of Preparation Examples B-1, B-2 and B-3, with the exception that, in the Tables 4, 5 and 6 given in Preparation Examples B-1, B-2 and B-3, 40 mg of atorvastatin calcium was used instead of 10.4 mg of rosuvastatin calcium, and the amounts of the other ingredients of the tablet part were doubled.

### Preparation Example B-8

### Preparation of Atorvastatin Calcium Mini-Tablets

Three types of coated mini-tablets were respectively prepared using the methods of Preparation Examples B-1, B-2 and B-3, with the exception that, in the Tables 4, 5 and 6 given in Preparation Examples B-1, B-2 and B-3, 10 mg of atorvastatin calcium was used instead of 10.4 mg of rosuvastatin calcium, and the amounts of the other ingredients of the tablet part were decreased by ½.

### Preparation Example B-9

### Preparation of 2 mg Pitavastatin Mini-Tablets

Three types of coated mini-tablets were respectively prepared using the methods of Preparation Examples B-1, B-2 and B-3, with the exception that, in the Tables 4, 5 and 6 given in Preparation Examples B-1, B-2 and B-3, 2 mg of pitavastatin was used instead of 10.4 mg of rosuvastatin calcium.

For reference, the above preparation is possible even upon using a pharmaceutically acceptable calcium or other salts of pitavastatin to be set as an amount of 2 mg of pitavastatin, in lieu of the pitavastatin used in this preparation example.

### Preparation Example B-10

### Preparation of 1 mg Pitavastatin Mini-Tablets

Three types of coated mini-tablets were respectively prepared using the methods of Preparation Examples B-1, B-2 and B-3, with the exception that, in the Tables 4, 5 and 6 given in Preparation Examples B-1, B-2 and B-3, 1 mg of pitavastatin was used instead of 10.4 mg of rosuvastatin calcium.

For reference, the above preparation is possible even upon using a pharmaceutically acceptable calcium or other salts of pitavastatin to be set as an amount of 1 mg of pitavastatin, in lieu of the pitavastatin used in this preparation example.

### Preparation Example B-11

### Preparation of 4 mg Pitavastatin Mini-Tablets

Three types of coated mini-tablets were respectively prepared using the methods of Preparation Examples B-1, B-2 and B-3, with the exception that, in the Tables 4, 5 and 6 given in Preparation Examples B-1, B-2 and B-3, 4 mg of pitavastatin was used instead of 10.4 mg of rosuvastatin calcium.

For reference, the above preparation is possible even upon using a pharmaceutically acceptable calcium or other salts of pitavastatin to be set as an amount of 4 mg of pitavastatin, in lieu of the pitavastatin used in this preparation example.

### Preparation Example B-12

### Preparation of 5 mg Pravastatin Mini-Tablets

Three types of coated mini-tablets were respectively prepared using the methods of Preparation Examples B-1, B-2 and B-3, with the exception that, in the Tables 4, 5 and 6 given in Preparation Examples B-1, B-2 and B-3, 5 mg of pravastatin was used instead of 10.4 mg of rosuvastatin calcium, and the amounts of the other ingredients of the tablet part were decreased by ½.

For reference, the above preparation is possible even upon using a pharmaceutically acceptable sodium or other salts of pravastatin to be set as an amount of 5 mg of pravastatin, in lieu of the pravastatin used in this preparation example.

### Preparation Example B-13

### Preparation of 10 mg Pravastatin Mini-Tablets

Three types of coated mini-tablets were respectively prepared using the methods of Preparation Examples B-1, B-2 and B-3, with the exception that, in the Tables 4, 5 and 6 given in Preparation Examples B-1, B-2 and B-3, 10 mg of pravastatin was used instead of 10.4 mg of rosuvastatin calcium, and the amounts of the other ingredients of the tablet part were not changed.

For reference, the above preparation is possible even upon using a pharmaceutically acceptable sodium or other salts of pravastatin to be set as an amount of 10 mg of pravastatin, in lieu of the pravastatin used in this preparation example.

### Preparation Example B-14

### Preparation of 20 mg Pravastatin Mini-Tablets

Three types of coated mini-tablets were respectively prepared using the methods of Preparation Examples B-1, B-2 and B-3, with the exception that, in the Tables 4, 5 and 6 given in Preparation Examples B-1, B-2 and B-3, 20 mg of pravastatin was used instead of 10.4 mg of rosuvastatin calcium, and the amounts of the other ingredients of the tablet part were doubled.

For reference, the above preparation is possible even upon using a pharmaceutically acceptable sodium or other salts of pravastatin to be set as an amount of 20 mg of pravastatin, in lieu of the pravastatin used in this preparation example.

### Preparation Example B-15

### Preparation of 40 mg Pravastatin Mini-Tablets

Three types of coated mini-tablets were respectively prepared using the methods of Preparation Examples B-1, B-2 and B-3, with the exception that, in the Tables 4, 5 and 6 given in Preparation Examples B-1, B-2 and B-3, 40 mg of pravastatin was used instead of 10.4 mg of rosuvastatin calcium, and the amounts of the other ingredients of the tablet part were increased four times.

For reference, the above preparation is possible even upon using a pharmaceutically acceptable sodium or other salts of pravastatin to be set as an amount of 40 mg of pravastatin, in lieu of the pravastatin used in this preparation example.

### EXAMPLE

The mini-tablets or mini-pellets containing the antithrombotic agent in the amount given in Preparation Example A and the mini-tablets containing the cholesterol lowering agent in the amount given in Preparation Example B were charged in No. 2, 1 or 0 capsules using a capsule filling machine (SF-100, available from Sejong Pharmatech).

### Example 1

### Capsules Containing Aspirin Mini-Pellets and Rosuvastatin Calcium Mini-Tablets

The enteric coated aspirin mini-pellets of Preparation Example A-3 and the coated rosuvastatin calcium mini-tablets of Preparation Example B-2 were placed in individual hard gelatin capsules (available from Suheung Capsule), thus manufacturing the title capsules.

35 spherical enteric coated aspirin mini-pellets having a diameter of 0.5˜2.0 mm and 20 circular coated rosuvastatin calcium mini-tablets having a diameter of 1.5˜2.5 mm were included per capsule.

### Example 2

### Capsules Containing Aspirin Mini-Pellets and Atorvastatin Calcium Mini-Tablets

The enteric coated aspirin mini-pellets of Preparation Example A-3 and the coated atorvastatin calcium mini-tablets prepared using the method of Preparation Example B-2 in Preparation Example B-6 were placed in individual hard gelatin capsules (available from Suheung Capsule), thus manufacturing the title capsules.

35 spherical enteric coated aspirin mini-pellets having a diameter of 0.5˜2.0 mm and 20 circular coated atorvastatin mini-tablets having a diameter of 1.5˜2.5 mm were included per capsule.

### Example 3

### Capsules Containing Aspirin Mini-Pellets and Pitavastatin Mini-Tablets

The enteric coated aspirin mini-pellets of Preparation Example A-3 and the coated pitavastatin mini-tablets prepared using the method of Preparation Example B-2 in Preparation Example B-11 were placed in individual hard gelatin capsules (available from Suheung Capsule), thus manufacturing the title capsules.

35 spherical enteric coated aspirin mini-pellets having a diameter of 0.5˜2.0 mm and 20 circular coated pitavastatin mini-tablets having a diameter of 1.5˜2.5 mm were included per capsule.

### Example 4

### Capsules Containing Aspirin Mini-Pellets and Pravastatin Mini-Tablets

The enteric coated aspirin mini-pellets of Preparation Example A-3 and the coated pravastatin mini-tablets prepared using the method of Preparation Example B-2 in Preparation Example B-12 were placed in individual hard gelatin capsules (available from Suheung Capsule), thus manufacturing the title capsules.

35 spherical enteric coated aspirin mini-pellets having a diameter of 0.5˜2.0 mm and 20 circular coated pravastatin mini-tablets having a diameter of 1.5˜2.5 mm were included per capsule.

### Example 5

### Capsules Containing Aspirin Mini-Tablets and Rosuvastatin Calcium Mini-Tablets

The enteric coated aspirin mini-tablets of Preparation Example A-2 and the coated rosuvastatin calcium mini-tablets of Preparation Example B-2 were placed in individual hard gelatin capsules (available from Suheung Capsule), thus manufacturing the title capsules.

20 circular enteric coated aspirin mini-tablets having a diameter of 1.5˜2.5 mm and 20 circular coated rosuvastatin calcium mini-tablets having a diameter of 1.5˜2.5 mm were included per capsule.

### Example 6

### Capsules Containing Aspirin Mini-Tablets and Atorvastatin Calcium Mini-Tablets

The enteric coated aspirin mini-tablets of Preparation Example A-2 and the coated atorvastatin calcium mini-tablets prepared using the method of Preparation Example B-2 in Preparation Example B-6 were placed in individual hard gelatin capsules (available from Suheung Capsule) thus manufacturing the title capsules.

20 circular enteric coated aspirin mini-tablets having a diameter of 1.5˜2.5 mm and 20 circular coated atorvastatin mini-tablets having a diameter of 1.5˜2.5 mm were included per capsule.

### Example 7

### Capsules Containing Aspirin Mini-Tablets and Pitavastatin Mini-Tablets

The enteric coated aspirin mini-tablets of Preparation Example A-2 and the coated pitavastatin mini-tablets prepared using the method of Preparation Example B-2 in Preparation Example B-11 were placed in individual hard gelatin capsules (available from Suheung Capsule) thus manufacturing the title capsules.

20 circular enteric coated aspirin mini-tablets having a diameter of 1.5˜2.5 mm and 20 circular coated pitavastatin mini-tablets having a diameter of 1.5˜2.5 mm were included per capsule.

### Example 8

### Capsules Containing Aspirin Mini-Tablets and Pravastatin Mini-Tablets

The enteric coated aspirin mini-tablets of Preparation Example A-2 and the coated pravastatin mini-tablets prepared using the method of Preparation Example B-2 in Preparation Example B-12 were placed in individual hard gelatin capsules (available from Suheung Capsule) thus manufacturing the title capsules.

20 circular enteric coated aspirin mini-pellets having a diameter of 1.5˜2.5 mm and 20 circular coated pravastatin mini-tablets having a diameter of 1.5˜2.5 mm were included per capsule.

### COMPARATIVE EXAMPLE

Compared to the examples of the present invention, the following comparative examples were prepared as below.

### Comparative Example 1

### Combined Tablets of Aspirin and Rosuvastatin Calcium

(1) Preparation of Aspirin Core

Aspirin and microcrystalline cellulose were mixed in the amounts shown in the Table 7 below using a high speed mixer (available from Sejong Pharmatech), and granulated with a previously prepared binder solution (a solution of hydroxypropylcellulose dispersed in purified water). The granules thus obtained were extruded using an extruder (available from Sejong Pharmatech), and spheronized using a Marumerizer (available from Sejong Pharmatech) thus obtaining spherical pellets.

(2) Enteric Coating of Aspirin Pellets

Pharmaceutically acceptable enteric coating materials (hydroxypropyl methylcellulose phthalate, diethyl phthalate, wheat starch and magnesium stearate) were mixed in the amounts shown in the Table 7 below, dissolved in ethanol and methylene chloride solvents, and then applied onto the above pellets using a flow coater (available from Glatt, Germany) to coat the above pellets.

(3) Production of Combined Tablets

Along with the above enteric coated aspirin pellets, rosuvastatin calcium, microcrystalline cellulose, calcium phosphate, anhydrous lactose, crospovidone and magnesium stearate were mixed in the amounts of the Table 7 below using a V-mixer, and then formed into tablets using a tablet press (KT-10S, available from KeumSung Machinery), after which the tablets were subjected to Opadry coating, thus obtaining final coated tablets.

TABLE 7

| | | | Amount |
|---|---|---|---|
| Components | | Ingredients | (mg) |
| Enteric | Core | Aspirin | 100 |
| Coated | layer | Microcrystalline Cellulose | 6.4 |
| Aspirin | | (PH101) | |
| Pellets | | Hydroxypropylcellulose | 4.4 |
| | | Purified water | 0.3 |
| | Coating | Hydroxypropyl Methylcellulose | 8.6 |
| | layer | Phthalate | |
| | | Diethyl Phthalate | 0.74 |
| | | Wheat Starch | 0.27 |
| | | Magnesium Stearate | 0.27 |
| | | Ethanol | 48 |
| | | Methylene Chloride | 90 |
| Rosuvastatin | Granule | Rosuvastatin Calcium | 10.4 |
| Calcium | part | Microcrystalline Cellulose | 68.0 |
| Granules | | Calcium Phosphate | 26.0 |
| | | Anhydrous Lactose | 22.1 |
| | | Crospovidone | 11.0 |
| | | Magnesium Stearate | 2.5 |
| | Coating | Opadry Pink (03B) | 7.0 |
| | layer | | |

### Comparative Example 2

### Combined Capsules of Enteric Coated Aspirin Pellets and Rosuvastatin Pellets

(1) Preparation of Aspirin Core

Aspirin and microcrystalline cellulose were mixed in the amounts shown in the Table 8 below using a high speed mixer (available from Sejong Pharmatech), and granulated with a previously prepared binder solution (a solution of hydroxypropylcellulose dispersed in purified water). The granules thus obtained were extruded using an extruder (available from Sejong Pharmatech), and spheronized using a Marumerizer (available from Sejong Pharmatech) thus obtaining spherical pellets.

(2) Enteric Coating of Aspirin Pellets

Pharmaceutically acceptable enteric coating materials (hydroxypropyl methylcellulose phthalate, diethyl phthalate, wheat starch and magnesium stearate) were mixed in the amounts of the Table 8 below, dissolved in ethanol and methylene chloride solvents, and then applied onto the above pellets using a flow coater (available from Glatt, Germany) to coat the above pellets.

(3) Preparation of Rosuvastatin Calcium Pellets

Hydroxymethylpropylcellulose 2910, rosuvastatin calcium, and calcium phosphate were dissolved in purified water in the amounts shown in the Table 8 below, and then applied onto inert sugar particles using a flow coater (available from Glatt, Germany) to coat the sugar pellets, after which the coated pellets were subjected to secondary coating using Opadry.

(4) Production of Combined Capsules of Rosuvastatin Calcium Pellets and Enteric Coated Aspirin Pellets

The enteric coated aspirin pellets and the rosuvastatin calcium pellets were charged into gelatin capsules (available from Suheung capsule).

TABLE 8

| | | | Amount |
|---|---|---|---|
| Components | | Ingredients | (mg) |
| Enteric | Core | Aspirin | 100 |
| Coated | layer | Microcrystalline Cellulose | 6.4 |
| Aspirin | | (PH101) | |
| Pellets | | Hydroxypropylcellulose | 4.4 |
| | | Purified water | 0.3 |
| | Coating | Hydroxypropyl Methylcellulose | 8.6 |
| | layer | Phthalate | |
| | | Diethyl Phthalate | 0.74 |
| | | Wheat Starch | 0.27 |
| | | Magnesium Stearate | 0.27 |
| | | Ethanol | 48 |
| | | Methylene Chloride | 90 |
| Rosuvastatin | Core | Inert Sugar Particles | 41.46 |
| Calcium | layer | Rosuvastatin Calcium | 10.4 |
| Pellets | | Hydroxymethylpropylcellulose | 15 |
| | | 2910 | |
| | | Calcium Phosphate | 26.0 |
| | Coating | Opadry Pink (03B) | 7.5 |
| | layer | | |

### Comparative Example 3

### Combined Tablets of Aspirin and Atorvastatin

Combined tablets of aspirin and atorvastatin were produced using the same ingredients and method as in Comparative Example 1, with the exception that 20 mg of atorvastatin was used instead of 10.4 mg of rosuvastatin calcium.

### Comparative Example 4

### Combined Capsules of Enteric Coated Aspirin Pellets and Atorvastatin Pellets

Combined capsules of enteric coated aspirin pellets and atorvastatin pellets were produced using the same ingredients and method as in Comparative Example 2, with the exception that 20 mg of atorvastatin was used instead of 10.4 mg of rosuvastatin calcium.

### Comparative Example 5

### Combined Tablets of Aspirin and Pitavastatin

Combined tablets of aspirin and pitavastatin were produced using the same ingredients and method as in Comparative Example 1, with the exception that 2 mg of pitavastatin was used instead of 10.4 mg of rosuvastatin calcium.

### Comparative Example 6

### Combined Capsules of Enteric Coated Aspirin Pellets and Pitavastatin Pellets

Combined capsules of enteric coated aspirin pellets and pitavastatin pellets were produced using the same ingredients and method as in Comparative Example 2, with the exception that 2 mg of pitavastatin was used instead of 10.4 mg of rosuvastatin calcium.

### Comparative Example 7

### Combined Tablets of Aspirin and Pravastatin

Combined tablets of aspirin and pravastatin were produced using the same ingredients and method as in Comparative Example 1, with the exception that 10 mg of pravastatin was used instead of 10.4 mg of rosuvastatin calcium.

### Comparative Example 8

### Combined Capsules of Enteric Coated Aspirin Pellets and Pravastatin Pellets

Combined capsules of enteric coated aspirin pellets and pravastatin pellets were produced using the same ingredients and method as in Comparative Example 2, with the exception that 10 mg of pravastatin was used instead of 10.4 mg of rosuvastatin calcium.

### TEST EXAMPLE

To compare the effects of the examples according to the present invention and the comparative examples, the following tests were conducted.

### Test Example 1

### (1) Comparison of Rosuvastatin Calcium Dissolution Rates

The rosuvastatin calcium dissolution rates (%) of the capsules of Example 1, the tablets of Comparative Example 1 and the capsules of Comparative Example 2 were measured (Table 9). The dissolution test was conducted using 900 ml of a dissolution testing liquid at 50 rpm for 30 min according to the second method of Dissolution Test of General Tests of Korea Pharmacopoeia. As such, the dissolution rate should be 93% or more. The dissolution testing liquid was prepared by dissolving 147 g of sodium citrate dihydrate in 2 L of deionized water, adding 3.3 g of anhydrous citric acid, and adding water to bring the total volume to 10 L. The pH of the solution was adjusted to 6.6±0.05 using sodium citrate or citric acid, as necessary.

TABLE 9
Rosuvastatin Dissolution Rate

| Dissolution Time/ | Dissolution Rate (%) | | |
|---|---|---|---|
| n = 12 (average) | Ex. 1 | C. Ex. 1 | C. Ex. 2 |
| 30 min | 92.6 ± 0.9% | 93.3 ± 3.4% | 94.3 ± 1.2% |

As is apparent from the above table, the rosuvastatin calcium dissolution rate of Example 1 was equivalent to the dissolution rates of Comparative Examples 1 and 2. When comparing the deviation of the dissolution rate of Example 1 with the deviations of the dissolution deviations of Comparative Examples 1 and 2, the deviation of Example 1 was 0.9%, which is comparatively low, and the deviations of Comparative Examples 1 and 2 were respectively 3.4% and 1.2% which are small. These deviations are considered to occur during the coating of inert particles, the filling of capsules, or the compression into tablets. In contrast, in the present example wherein the plurality of mini-tablets prepared by using the multi-tip punches is charged in one capsule, the deviation was lower than that of a single unitary tablet or unitary capsule.

### (2) Comparison of Aspirin Dissolution Rates

The aspirin dissolution rates (%) of the capsules of Example 1, the tablets of Comparative Example 1 and the capsules of Comparative Example 2 were measured (Table 10). The standards were based on USP 28 ‘Aspirin Delayed Release Capsule’.

TABLE 10
Aspirin Dissolution Rate

| Dissolution Time/ | Dissolution Rate (%) | | |
|---|---|---|---|
| n = 12 (average) | Ex. 1 | C. Ex. 1 | C. Ex. 2 |
| 120 min (pH 1.2) | 0.9 ± 0.2% | 5.8 ± 4.3% | 1.4 ± 0.3% |
| 90 min (pH 6.8) | 94.9 ± 1.2% | 92.2 ± 0.8% | 93.9 ± 0.9% |

As is apparent from the above table, the dissolution standards of USP 28 ‘Aspirin Delayed Release Capsule’, that is, 10% or less at pH 1.2 for 120 min and 80% or more at pH 6.8 for 90 min were satisfied in Example 1 and Comparative Examples 1 and 2.

In the acid resistance test, at pH 1.2, the dissolution of Comparative Example 1 was higher than that of Example 1 and the deviation thereof was larger. This is considered to be because the enteric coating is partially damaged due to the friction between adjacent enteric aspirin pellets in the tablet press and the high pressure of the tablet press in the compression procedure upon tableting the mixture of aspirin pellets and rosuvastatin calcium. Meanwhile, Comparative Example 2 was not significantly different from Example 1.

At pH 6.8, the dissolution rates of Example 1 including the plurality of mini-pellets and of Comparative Examples 1 and 2 including the plurality of mini-pellets were not significantly different.

### (3) Comparison of the Stability of Formulations

The stability of each of the capsules of Example 1, the tablets of Comparative Example 1 and the capsules of Comparative Example 2 was tested for 6 months under the following conditions: long storage test conditions: 25° C., 60% RH, acceleration test conditions: 40° C., 75% RH. Respective amounts were measured using Alliance HPLC (High Performance Liquid Chromatography, available from Water). The results are shown in the Table 11 below (initial amounts of aspirin and rosuvastatin of Example 1 and Comparative Examples 1 and 2 used in the long storage test and the acceleration test) and Table 12 below (amounts of aspirin and rosuvastatin of Example 1 and Comparative Examples 1 and 2 after 6-month stability test).

TABLE 11
Initial Amounts of Aspirin and Rosuvastatin

| | Amount (%) | | |
|---|---|---|---|
| Initial | Ex. 1 | C. Ex. 1 | C. Ex. 2 |
| Aspirin | 100.1 | 100.6 | 100.1 |
| Rosuvastatin Calcium | 99.8 | 98.3 | 99.7 |

TABLE 12
Amounts of Aspirin and Rosuvastatin after 6-Month Stability Test

Amount (%)

| After 6 months | | Ex. 1 | C. Ex. 1 | C. Ex. 2 |
|---|---|---|---|---|
| Long Storage | Aspirin | 100.4 | 98.1 | 100.2 |
| | Rosuvastatin | 99.8 | 98.3 | 91.7 |
| | Calcium | | | |
| Acceleration | Aspirin | 98.3 | 92.6 | 99.0 |
| | Rosuvastatin | 98.8 | 90.1 | 85.3 |
| | Calcium | | | |

As is apparent from the Table 12, the results of the 6-month stability test were that the amount of the capsules of Example 1 underwent no significant change for 6 months and maintained stability. However, in Comparative Examples 1 and 2, the amounts were remarkably decreased upon 6-month acceleration testing, and these formulations were identified as being unstable.

### Test Example 2

### (1) Comparison of Rosuvastatin Calcium Dissolution Rates

The rosuvastatin calcium dissolution rates (%) of the capsules of Example 5, the tablets of Comparative Example 1 and the capsules of Comparative Example 2 were measured in the same manner as in Test Example 1 (Table 13).

TABLE 13
Rosuvastatin Dissolution Rate

| Dissolution Time/ | Dissolution Rate (%) | | |
|---|---|---|---|
| n = 12 (average) | Ex. 5 | C. Ex. 1 | C. Ex. 2 |
| 30 min | 94.2 ± 0.9% | 92.5 ± 2.9% | 93.2 ± 1.5% |

As is apparent from the above table, the rosuvastatin calcium dissolution rate of Example 5 was equivalent to the dissolution rates of Comparative Examples 1 and 2. When comparing the deviation of the dissolution rate of Example 5 with the deviations of the dissolution rates of Comparative Examples 1 and 2, the deviation of Example 5 was 0.9% which is comparatively low, whereas the deviations of Comparative Examples 1 and 2 were respectively 2.9% and 1.5% which are small. These deviations are considered to occur during the coating of inert particles, the filling of capsules or the compression into tablets. In contrast, in the present example wherein the plurality of mini-tablets prepared by using the multi-tip punches is charged in one capsule, the deviation was lower than that of a single unitary tablet or unitary capsule.

### (2) Comparison of Aspirin Dissolution Rates

The aspirin dissolution rates (%) of the capsules of Example 5, the tablets of Comparative Example 1 and the capsules of Comparative Example 2 were measured (Table 14). The standards were based on USP 28 ‘Aspirin Delayed Release Capsule’.

TABLE 14
Aspirin Dissolution Rate

| Dissolution Time/ | Dissolution Rate (%) | | |
|---|---|---|---|
| n = 12 (average) | Ex. 5 | C. Ex. 1 | C. Ex. 2 |
| 120 min (pH 1.2) | 0.6 ± 0.3% | 4.9 ± 3.8% | 0.9 ± 0.3% |
| 90 min (pH 6.8) | 93.8 ± 1.1% | 94.1 ± 1.3% | 92.7 ± 1.2% |

As is apparent from the above table, the dissolution standards of USP 28 ‘Aspirin Delayed Release Capsule’, that is, 10% or less at pH 1.2 for 120 min and 80% or more at pH 6.8 for 90 min were satisfied in Example 1 and Comparative Examples 1 and 2.

In the acid resistance test, at pH 1.2, the dissolution of Comparative Example 1 was higher than that of Example 5 and the deviation thereof was larger. This is considered to be because the enteric coating is partially damaged due to the friction between adjacent enteric aspirin pellets in the tablet press and the high pressure of the tablet press in the compression procedure upon tableting the mixture of aspirin pellets and rosuvastatin calcium. Meanwhile, Comparative Example 2 was not significantly different from Example 5.

At pH 6.8, the dissolution rates of Example 5 including the plurality of mini-tablets and of Comparative Examples 1 and 2 including the plurality of mini-pellets were not significantly different.

### (3) Comparison of the Stability of Formulations

The stability of each of the capsules of Example 5, the tablets of Comparative Example 1 and the capsules of Comparative Example 2 was tested for 6 months under the following conditions: long storage test conditions: 25° C., 60% RH, acceleration test conditions: 40° C., 75% RH. Respective amounts were measured using Alliance HPLC (available from Water). The results are shown in the Table 15 below (initial amounts of aspirin and rosuvastatin of Example 5 and Comparative Examples 1 and 2 used in the long storage test and the acceleration test) and the Table 16 below (amounts of aspirin and rosuvastatin of Example 5 and Comparative Examples 1 and 2 after a 6-month stability test).

TABLE 15
Initial Amounts of Aspirin and Rosuvastatin

| | Amount (%) | | |
|---|---|---|---|
| Initial | Ex. 5 | C. Ex. 1 | C. Ex. 2 |
| Aspirin | 100.3 | 100.4 | 100.1 |
| Rosuvastatin Calcium | 99.4 | 98.4 | 99.5 |

TABLE 16
Amounts of Aspirin and Rosuvastatin after 6-Month Stability Test

Amount (%)

| After 6 months | | Ex. 5 | C. Ex. 1 | C. Ex. 2 |
|---|---|---|---|---|
| Long Storage | Aspirin | 100.1 | 99.2 | 100.3 |
| | Rosuvastatin | 99.1 | 98.5 | 92.1 |
| | Calcium | | | |
| Acceleration | Aspirin | 98.4 | 92.9 | 98.7 |
| | Rosuvastatin | 97.9 | 89.7 | 84.1 |
| | Calcium | | | |

As is apparent from the Table 16, the results of the 6-month stability test were that the amount of the capsules of Example 5 underwent no significant change for 6 months and maintained stability. However, in Comparative Examples 1 and 2, the amounts were remarkably decreased upon 6-month acceleration testing, and these formulations were identified as being unstable.

### Test Example 3

### (1) Comparison of Atorvastatin Dissolution Rates

The atorvastatin dissolution rates (%) of the capsules of Example 2, the tablets of Comparative Example 3 and the capsules of Comparative Example 4 were measured (Table 17). The dissolution test was conducted using 900 ml of a dissolution testing liquid at 50 rpm for 30 min according to the second method of Dissolution Test of General Tests of Korea Pharmacopoeia. As such, the dissolution rate should be 80% or more.
Dissolution testing liquid: water
Test liquid: after the dissolution test, 20 ml of the dissolution liquid was taken, filtered and used as a test liquid.
Standard solution: 50 mg of an atorvastatin standard product was dissolved in a solution (water/ACN=1:1), and 5 ml of this solution was mixed with water so that the total amount of the solution was exactly adjusted to 200 ml, and the resulting solution was used as a standard solution.
Measurement condition: 244 nm

TABLE 17
Atorvastatin Dissolution Rate

| Dissolution Time/ | Dissolution Rate (%) | | |
|---|---|---|---|
| n = 12 (average) | Ex. 2 | C. Ex. 3 | C. Ex. 4 |
| 30 min | 91.1 ± 1.1% | 90.7 ± 2.9% | 91.3 ± 1.7% |

As is apparent from the above table, the atorvastatin dissolution rate of Example 2 was equivalent to the dissolution rates of Comparative Examples 3 and 4. When comparing the deviation of the dissolution rate of Example 2 with the deviations of the dissolution rates of Comparative Examples 3 and 4, the deviation of Example 2 was 1.1% which is comparatively low, whereas the deviations of Comparative Examples 3 and 4 were respectively 2.9% and 1.7% which are small. These deviations are considered to occur during the coating of inert particles, the filling of capsules, or the compression into tablets. In contrast, in the present example wherein the plurality of mini-tablets prepared by using the multi-tip punches is charged in one capsule, the deviation was lower than that of a single unitary tablet or unitary capsule.

### (2) Comparison of Aspirin Dissolution Rates

The aspirin dissolution rates (%) of the capsules of Example 2, and Comparative Examples 3 and 4 were measured (Table 18). The standards were based on USP 28 ‘Aspirin Delayed Release Capsule’.

TABLE 18
Aspirin Dissolution Rate

| Dissolution Time/ | Dissolution Rate (%) | | |
|---|---|---|---|
| n = 12 (average) | Ex. 2 | C. Ex. 3 | C. Ex. 4 |
| 120 min (pH 1.2) | 0.7 ± 0.1% | 4.3 ± 3.7% | 1.6 ± 0.5% |
| 90 min (pH 6.8) | 93.7 ± 0.9% | 94.0 ± 1.7% | 93.5 ± 0.6% |

As is apparent from the above table, the dissolution standards of USP 28 ‘Aspirin Delayed Release Capsule’, that is, 10% or less at pH 1.2 for 120 min and 80% or more at pH 6.8 for 90 min were satisfied in Example 2 and Comparative Examples 3 and 4.

In the acid resistance test, at pH 1.2, the dissolution of Comparative Example 3 was higher than that of Example 2 and the deviation thereof was larger. This is considered to be because the enteric coating is partially damaged due to friction between adjacent enteric aspirin pellets in the tablet press and the high pressure of the tablet press in the compression procedure upon tableting the mixture of aspirin pellets and atorvastatin. Meanwhile, Comparative Example 4 was not significantly different from Example 2.

At pH 6.8, the dissolution rates of Example 2 including the plurality of mini-pellets and of Comparative Examples 3 and 4 including the plurality of mini-pellets were not significantly different.

### (3) Comparison of the Stability of Formulations

The stability of each of the capsules of Example 2, the tablets of Comparative Example 3 and the capsules of Comparative Example 4 was tested for 6 months under the following conditions: long storage test conditions: 25° C., 60% RH, acceleration test conditions: 40° C., 75% RH. Respective amounts were measured using Alliance HPLC (available from Water). The results are shown in the Table 19 below (initial amounts of aspirin and atorvastatin of Example 2 and Comparative Examples 3 and 4 used in the long storage test and the acceleration test) and the Table 20 below (amounts of aspirin and atorvastatin of Example 2 and Comparative Examples 3 and 4 after a 6-month stability test).

TABLE 19
Initial Amounts of Aspirin and Atorvastatin

| | Amount (%) | | |
|---|---|---|---|
| Initial | Ex. 2 | C. Ex. 3 | C. Ex. 4 |
| Aspirin | 100.3 | 100.2 | 99.9 |
| Atorvastatin | 98.9 | 99.1 | 99.5 |

TABLE 20
Amounts of Aspirin and Atorvastatin after 6-Month Stability Test

Amount (%)

| After 6 months | | Ex. 2 | C. Ex. 3 | C. Ex. 4 |
|---|---|---|---|---|
| Long Storage | Aspirin | 99.3 | 99.7 | 100.1 |
| | Atorvastatin | 97.9 | 98.1 | 93.6 |
| Acceleration | Aspirin | 98.1 | 93.1 | 98.3 |
| | Atorvastatin | 96.2 | 89.8 | 87.1 |

As is apparent from the Table 20, the results of the 6-month stability test were that the amount of the capsules of Example 2 underwent no significant change for 6 months and maintained stability. However, in Comparative Examples 3 and 4, the amounts were remarkably decreased upon 6-month acceleration testing, and these formulations were identified as being unstable.

### Test Example 4

### (1) Comparison of Atorvastatin Dissolution Rates

The atorvastatin dissolution rates (%) of the capsules of Example 6, the tablets of Comparative Example 3 and the capsules of Comparative Example 4 were measured in the same manner as in Test Example 3 (Table 21).

TABLE 21
Atorvastatin Dissolution Rate

| Dissolution Time/ | Dissolution Rate (%) | | |
|---|---|---|---|
| n = 12 (average) | Ex. 6 | C. Ex. 3 | C. Ex. 4 |
| 30 min | 92.3 ± 0.9% | 91.2 ± 3.1% | 93.0 ± 1.6% |

As is apparent from the above table, the atorvastatin dissolution rate of Example 6 was equivalent to the dissolution rates of Comparative Examples 3 and 4. When comparing the deviation of the dissolution rate of Example 6 with the deviations of the dissolution rates of Comparative Examples 3 and 4, the deviation of Example 6 was 0.9% which is comparatively low, whereas the deviations of Comparative Examples 3 and 4 were respectively 3.1% and 1.6% which are small. These deviations are considered to be formed during the coating of inert particles, the filling of capsules or the compression into tablets. In contrast, in the present example wherein the plurality of mini-tablets prepared by using the multi-tip punches is charged in one capsule, the deviation was lower than that of a single unitary tablet or unitary capsule.

### (2) Comparison of Aspirin Dissolution Rates

The aspirin dissolution rates (%) of the capsules of Example 6, and Comparative Examples 3 and 4 were measured (Table 22). The standards were based on USP 28 ‘Aspirin Delayed Release Capsule’.

TABLE 22
Aspirin Dissolution Rate

| Dissolution Time/ | Dissolution Rate (%) | | |
|---|---|---|---|
| n = 12 (average) | Ex. 6 | C. Ex. 3 | C. Ex. 4 |
| 120 min (pH 1.2) | 0.9 ± 0.2% | 4.1 ± 3.9% | 1.9 ± 0.8% |
| 90 min (pH 6.8) | 94.1 ± 0.8% | 93.1 ± 1.7% | 92.4 ± 0.8% |

As is apparent from the above table, the dissolution standards of USP 28 ‘Aspirin Delayed Release Capsule’, that is, 10% or less at pH 1.2 for 120 min and 80% or more at pH 6.8 for 90 min were satisfied in Example 6 and Comparative Examples 3 and 4.

In the acid resistance test, at pH 1.2, the dissolution of Comparative Example 3 was higher than that of Example 6 and the deviation thereof was larger. This is considered to be because the enteric coating is partially damaged due to the friction between adjacent enteric aspirin pellets in the tablet press and the high pressure of the tablet press in the compression procedure upon tableting the mixture of aspirin pellets and atorvastatin. Meanwhile, Comparative Example 4 was not significantly different from Example 6.

At pH 6.8, the dissolution rates of Example 6 including the plurality of mini-tablets and of Comparative Examples 3 and 4 including the plurality of mini-pellets were not significantly different.

### (3) Comparison of the Stability of Formulations

The stability of each of capsules of Example 6, the tablets of Comparative Example 3 and the capsules of Comparative Example 4 was tested for 6 months under the following conditions: long storage test conditions: 25° C., 60% RH, acceleration test conditions: 40° C., 75% RH. Respective amounts were measured using Alliance HPLC (available from Water). The results are shown in the Table 23 below (initial amounts of aspirin and atorvastatin of Example 6 and Comparative Examples 3 and 4 used in the long storage test and the acceleration test) and the Table 24 below (amounts of aspirin and atorvastatin of Example 6 and Comparative Examples 3 and 4 after a 6-month stability test).

TABLE 23
Initial Amounts of Aspirin and Atorvastatin

| | Amount (%) | | |
|---|---|---|---|
| Initial | Ex. 6 | C. Ex. 3 | C. Ex. 4 |
| Aspirin | 100.1 | 100.3 | 100.5 |
| Atorvastatin | 98.8 | 98.9 | 99.3 |

TABLE 24
Amounts of Aspirin and Atorvastatin after 6-Month Stability Test

Amount (%)

| After 6 months | | Ex. 6 | C. Ex. 3 | C. Ex. 4 |
|---|---|---|---|---|
| Long Storage | Aspirin | 99.5 | 99.6 | 100.0 |
| | Atorvastatin | 96.3 | 97.6 | 94.1 |
| Acceleration | Aspirin | 98.3 | 94.2 | 97.5 |
| | Atorvastatin | 96.6 | 89.1 | 88.4 |

As is apparent from the Table 24, the results of the 6-month stability test were that the amount of the capsules of Example 6 underwent no significant change for 6 months and maintained stability. However, in Comparative Examples 3 and 4, the amounts were remarkably decreased upon 6-month acceleration testing, and these formulations were identified as being unstable.

### Test Example 5

### (1) Comparison of Pitavastatin Dissolution Rates

The pitavastatin dissolution rates (%) of the capsules of Example 3, the tablets of Comparative Example 5 and the capsules of Comparative Example 6 were measured (Table 25). The dissolution test was conducted using a 900 ml of a dissolution testing liquid at 35 rpm for 45 min according to the Basket method of the first method of Dissolution Test of General Tests of Korea Pharmacopoeia. As such, the dissolution rate should be 85% or more.
Dissolution testing liquid

0.05M Phosphate Buffer (pH 6.8) solution

TABLE 25
Pitavastatin Dissolution Rate

| Dissolution Time/ | Dissolution Rate (%) | | |
|---|---|---|---|
| n = 12 (average) | Ex. 3 | C. Ex. 5 | C. Ex. 6 |

| | | | |
|---|---|---|---|
| 30 min | 91.8 ± 0.9% | 91.1 ± 3.2% | 93.0 ± 1.1% |

As is apparent from the above table, the pitavastatin dissolution rate of Example 3 was equivalent to the dissolution rates of Comparative Examples 5 and 6. When comparing the deviation of the dissolution rate of Example 3 with the deviations of the dissolution rates of Comparative Examples 5 and 6, the deviation of Example 3 was 0.9%, which is comparatively low, whereas the deviations of Comparative Examples 5 and 6 were respectively 3.2% and 1.1% which are small. These deviations are considered to be formed during the coating of inert particles, the filling of capsules or the compression into tablets. In contrast, in the present example wherein the plurality of mini-tablets prepared by using the multi-tip punches is charged in one capsule, the deviation was lower than that of a single unitary tablet or unitary capsule.

### (2) Comparison of Aspirin Dissolution Rates

The aspirin dissolution rates (%) of the capsules of Example 3, and Comparative Examples 5 and 6 were measured (Table 26). The standards were based on USP 28 ‘Aspirin Delayed Release Capsule’.

TABLE 26
Aspirin Dissolution Rate

| Dissolution Time/ | Dissolution Rate (%) | | |
|---|---|---|---|
| n = 12 (average) | Ex. 3 | C. Ex. 5 | C. Ex. 6 |
| 120 min (pH 1.2) | 0.9 ± 0.3% | 3.9 ± 3.5% | 1.4 ± 0.7% |
| 90 min (pH 6.8) | 94.1 ± 0.8% | 93.5 ± 1.6% | 93.8 ± 0.8% |

As is apparent from the above table, the dissolution standards of USP 28 ‘Aspirin Delayed Release Capsule’, that is, 10% or less at pH 1.2 for 120 min and 80% or more at pH 6.8 for 90 min were satisfied in Example 3 and Comparative Examples 5 and 6.

In the acid resistance test, at pH 1.2, the dissolution of Comparative Example 5 was higher than that of Example 3 and the deviation thereof was larger. This is considered to be because the enteric coating is partially damaged due to the friction between adjacent enteric aspirin pellets in the tablet press and the high pressure of the tablet press in the compression procedure upon tableting the mixture of aspirin pellets and pitavastatin. Meanwhile, Comparative Example 6 was not significantly different from Example 3.

At pH 6.8, the dissolution rates of Example 3 including the plurality of mini-pellets and of Comparative Examples 5 and 6 including the plurality of mini-pellets were not significantly different.

### (3) Comparison of the Stability of Formulations

The stability of each of the capsules of Example 3, the tablets of Comparative Example 5 and the capsules of Comparative Example 6 was tested for 6 months under the following conditions: long storage test conditions: 25° C., 60% RH, acceleration test conditions: 40° C., 75% RH. Respective amounts were measured using Alliance HPLC (available from Water). The results are shown in the Table 27 below (initial amounts of aspirin and pitavastatin of Example 3 and Comparative Examples 5 and 6 used in the long storage test and the acceleration test) and the Table 28 below (amounts of aspirin and pitavastatin of Example 3 and Comparative Examples 5 and 6 after a 6-month stability test).

TABLE 27
Initial Amounts of Aspirin and Pitavastatin

| | Amount (%) | | |
|---|---|---|---|
| Initial | Ex. 3 | C. Ex. 5 | C. Ex. 6 |
| Aspirin | 99.6 | 100.2 | 100.8 |
| Pitavastatin | 99.9 | 100.1 | 99.2 |

TABLE 28
Amounts of Aspirin and Pitavastatin after 6-Month Stability Test

| | | Amount (%) | | |
|---|---|---|---|---|
| After 6 months | | Ex. 3 | C. Ex. 5 | C. Ex. 6 |
| Long Storage | Aspirin | 99.2 | 96.1 | 99.7 |
| | Pitavastatin | 98.6 | 97.4 | 90.7 |
| Acceleration | Aspirin | 97.6 | 92.7 | 98.2 |
| | Pitavastatin | 98.4 | 88.5 | 84.2 |

As is apparent from the Table 28, the results of the 6-month stability test were that the capsules of Example 3 underwent no significant amount change for 6 months and maintained stability. However, in Comparative Examples 5 and 6, the amounts were remarkably decreased upon 6-month acceleration testing, and these formulations were identified as being unstable.

### Test Example 6

### (1) Comparison of Pitavastatin Dissolution Rates

The pitavastatin dissolution rates (%) of the capsules of Example 7, the tablets of Comparative Example 5 and the capsules of Comparative Example 6 were measured in the same manner as in Test Example 5 (Table 29).

TABLE 29
Pitavastatin Dissolution Rate

| Dissolution Time/ | Dissolution Rate (%) | | |
|---|---|---|---|
| n = 12 (average) | Ex. 7 | C. Ex. 5 | C. Ex. 6 |
| 30 min | 93.1 ± 0.8% | 92.4 ± 3.0% | 92.8 ± 1.3% |

As is apparent from the above table, the pitavastatin calcium dissolution rate of Example 7 was equivalent to the dissolution rates of Comparative Examples 5 and 6. When comparing the deviation of the dissolution rate of Example 7 with the deviations of the dissolution rates of Comparative Examples 5 and 6, the deviation of Example 7 was 0.8%, which is comparatively low, whereas the deviations of Comparative Examples 5 and 6 were respectively 3.0% and 1.3% which are small. These deviations are considered to be formed during the coating of inert particles, the filling of capsules or the compression into tablets. In contrast, in the present example wherein the plurality of mini-tablets prepared by using the multi-tip punches is charged in one capsule, the deviation was lower than that of a single unitary tablet or unitary capsule.

(2) Comparison of Aspirin Dissolution Rates

The aspirin dissolution rates (%) of the capsules of Example 7, and Comparative Examples 5 and 6 were measured (Table 30). The standards were based on USP 28 ‘Aspirin Delayed Release Capsule’.

TABLE 30
Aspirin Dissolution Rate

| Dissolution Time/ | Dissolution Rate (%) | | |
|---|---|---|---|
| n = 12 (average) | Ex. 7 | C. Ex. 5 | C. Ex. 6 |
| 120 min (pH 1.2) | 1.1 ± 0.4% | 3.1 ± 2.8% | 1.2 ± 0.8% |
| 90 min (pH 6.8) | 94.4 ± 0.9% | 93.6 ± 1.7% | 93.2 ± 0.7% |

As is apparent from the above table, the dissolution standards of USP 28 ‘Aspirin Delayed Release Capsule’, that is, 10% or less at pH 1.2 for 120 min and 80% or more at pH 6.8 for 90 min were satisfied in Example 7 and Comparative Examples 5 and 6.

In the acid resistance test, at pH 1.2, the dissolution of Comparative Example 5 was higher than that of Example 7 and the deviation thereof was larger. This is considered to be because the enteric coating is partially damaged due to the friction between adjacent enteric aspirin pellets in the tablet press and the high pressure of the tablet press in the compression procedure upon tableting the mixture of aspirin pellets and pitavastatin. Meanwhile, Comparative Example 6 was not significantly different from Example 7.

At pH 6.8, the dissolution rates of Example 7 including the plurality of mini-tablets and of Comparative Examples 5 and 6 including the plurality of mini-pellets were not significantly different.

### (3) Comparison of the Stability of Formulations

The stability of each of the capsules of Example 7, the tablets of Comparative Example 5 and the capsules of Comparative Example 6 was tested for 6 months under the following conditions: long storage test conditions: 25° C., 60% RH, acceleration test conditions: 40° C., 75% RH. Respective amounts were measured using Alliance HPLC (available from Water). The results are shown in the Table 31 below (initial amounts of aspirin and pitavastatin of Example 7 and Comparative Examples 5 and 6 used in the long storage test and the acceleration test) and the Table 32 below (amounts of aspirin and pitavastatin of Example 7 and Comparative Examples 5 and 6 after a 6-month stability test).

TABLE 31
Initial Amounts of Aspirin and Pitavastatin

| | Amount (%) | | |
|---|---|---|---|
| Initial | Ex. 7 | C. Ex. 5 | C. Ex. 6 |
| Aspirin | 99.3 | 99.4 | 99.5 |
| Pitavastatin | 98.3 | 98.9 | 99.6 |

TABLE 32
Amounts of Aspirin and Pitavastatin after 6-Month Stability Test

| | | Amount (%) | | |
|---|---|---|---|---|
| After 6 months | | Ex. 7 | C. Ex. 5 | C. Ex. 6 |
| Long Storage | Aspirin | 99.5 | 96.8 | 99.3 |
| | Pitavastatin | 98.1 | 97.2 | 89.5 |
| Acceleration | Aspirin | 97.3 | 93.1 | 97.9 |
| | Pitavastatin | 98.1 | 88.9 | 85.7 |

As is apparent from the Table 32, the results of the 6-month stability test were that the amount of the capsules of Example 7 underwent no significant change for 6 months and maintained stability. However, in Comparative Examples 5 and 6, the amounts were remarkably decreased upon 6-month acceleration testing, and these formulations were identified as being unstable.

### Test Example 7

### (1) Comparison of Pravastatin Dissolution Rates

The pravastatin dissolution rates (%) of the capsules of Example 4, the tablets of Comparative Example 7 and the capsules of Comparative Example 8 were measured (Table 33). The dissolution test was conducted using 900 ml of a dissolution testing liquid at 50 rpm for 30 min according to the second method of Dissolution Test of General Tests of Korea Pharmacopoeia. As such, the dissolution rate should be 85% or more.
Dissolution testing liquid: water
Test liquid: after the dissolution test, exactly 1 ml of a dissolution liquid was taken and mixed with 2 ml of the dissolution testing liquid and thus used as a test liquid.
Standard solution: 10 mg of a standard product was dissolved in 900 ml of a testing liquid, and 50 ml of the resulting solution was diluted with 100 ml of the dissolution test liquid.
Measurement wavelength: 239 nm

TABLE 33
Pravastatin Dissolution Rate

| Dissolution Time/ | Dissolution Rate (%) | | |
|---|---|---|---|
| n = 12 (average) | Ex. 4 | C. Ex. 7 | C. Ex. 8 |
| 30 min | 92.1 ± 1.1% | 91.6 ± 4.2% | 92.3 ± 1.6% |

As is apparent from the above table, the pravastatin dissolution rate of Example 4 was equivalent to the dissolution rates of Comparative Examples 7 and 8. When comparing the deviation of the dissolution rate of Example 4 with the deviations of the dissolution rates of Comparative Examples 7 and 8, the deviation of Example 4 was 1.1%, which is comparatively low, whereas the deviations of Comparative Examples 7 and 8 were respectively 4.2% and 1.6% which are small. These deviations are considered to be formed during the coating of inert particles, the filling of capsules or the compression into tablets. In contrast, in the present example wherein the plurality of mini-tablets prepared by using the multi-tip punches is charged in one capsule, the deviation was lower than that of a single unitary tablet or unitary capsule.

### (2) Comparison of Aspirin Dissolution Rates

The aspirin dissolution rates (%) of the capsules of Example 4, and Comparative Examples 7 and 8 were measured (Table 34). The standards were based on USP 28 ‘Aspirin Delayed Release Capsule’.

TABLE 34
Aspirin Dissolution Rate

| Dissolution Time/ | Dissolution Rate (%) | | |
|---|---|---|---|
| n = 12 (average) | Ex. 4 | C. Ex. 7 | C. Ex. 8 |
| 120 min (pH 1.2) | 0.8 ± 0.2% | 3.7 ± 3.3% | 1.3 ± 0.9% |
| 90 min (pH 6.8) | 95.2 ± 0.9% | 94.2 ± 1.5% | 93.6 ± 0.9% |

As is apparent from the above table, the dissolution standards of USP 28 ‘Aspirin Delayed Release Capsule’, that is, 10% or less at pH 1.2 for 120 min and 80% or more at pH 6.8 for 90 min were satisfied in Example 4 and Comparative Examples 7 and 8.

In the acid resistance test, at pH 1.2, the dissolution of Comparative Example 7 was higher than that of Example 4 and the deviation thereof was larger. This is considered to be because the enteric coating is partially damaged due to the friction between adjacent enteric aspirin pellets in the tablet press and the high pressure of the tablet press in the compression procedure upon tableting the mixture of aspirin pellets and pravastatin. Meanwhile, Comparative Example 8 was not significantly different from Example 4.

At pH 6.8, the dissolution rates of Example 4 including the plurality of mini-pellets and of Comparative Examples 7 and 8 including the plurality of mini-pellets were not significantly different.

### (3) Comparison of the Stability of Formulations

The stability of each of Example 4 and Comparative Examples 7 and 8 was tested for 6 months under the following conditions: long storage test conditions: 25° C., 60% RH, acceleration test conditions: 40° C., 75% RH. Respective amounts were measured using Alliance HPLC (available from Water). The results are shown in the Table 35 below (initial amounts of aspirin and pravastatin of Example 4 and Comparative Examples 7 and 8 used in the long storage test and the acceleration test) and the Table 36 below (amounts of aspirin and pravastatin of Example 4 and Comparative Examples 7 and 8 after a 6-month stability test).

TABLE 35
Initial Amounts of Aspirin and Pravastatin

| | Amount (%) | | |
|---|---|---|---|
| Initial | Ex. 4 | C. Ex. 7 | C. Ex. 8 |
| Aspirin | 99.8 | 98.1 | 99.5 |
| Pravastatin | 99.3 | 100.2 | 98.4 |

TABLE 36
Amounts of Aspirin and Pravastatin after 6-Month Stability Test

| | | Amount (%) | | |
|---|---|---|---|---|
| After 6 months | | Ex. 4 | C. Ex. 7 | C. Ex. 8 |
| Long Storage | Aspirin | 98.3 | 95.9 | 98.9 |
| | Pravastatin | 99.1 | 95.1 | 91.6 |
| Acceleration | Aspirin | 96.9 | 91.9 | 97.0 |
| | Pravastatin | 96.8 | 89.2 | 83.3 |

As is apparent from the Table 36, the results of the 6-month stability test were that the amount of the capsules of Example 4 underwent no significant change for 6 months and maintained stability. However, in Comparative Examples 7 and 8, the amounts were remarkably decreased upon 6-month acceleration testing, and these formulations were identified as being unstable.

### Test Example 8

### (1) Comparison of Pravastatin Dissolution Rates

The pravastatin dissolution rates (%) of the capsules of Example 8, the tablets of Comparative Example 7 and the capsules of Comparative Example 8 were measured in the same manner as in Test Example 7 (Table 37).

TABLE 37
Pravastatin Dissolution Rate

| Dissolution Time/ | Dissolution Rate (%) | | |
|---|---|---|---|
| n = 12 (average) | Ex. 8 | C. Ex. 7 | C. Ex. 8 |
| 30 min | 92.7 ± 1.0% | 92.6 ± 3.9% | 92.7 ± 1.5% |

As is apparent from the above table, the pravastatin dissolution rate of Example 8 was equivalent to the dissolution rates of Comparative Examples 7 and 8. When comparing the deviation of the dissolution rate of Example 8 with the deviations of the dissolution rates of Comparative Examples 7 and 8, the deviation of Example 8 was 1.0%, which is comparatively low, whereas the deviations of Comparative Examples 7 and 8 were respectively 3.9% and 1.5% which are small. These deviations are considered to be formed during the coating of inert particles, the filling of capsules or the compression into tablets. In contrast, in the present example wherein the plurality of mini-tablets prepared by using the multi-tip punches is charged in one capsule, the deviation was lower than that of a single unitary tablet or unitary capsule.

### (2) Comparison of Aspirin Dissolution Rates

The aspirin dissolution rates (%) of the capsules of Example 8, and Comparative Examples 7 and 8 were measured (Table 38). The standards were based on USP 28 ‘Aspirin Delayed Release Capsule’.

TABLE 38
Aspirin Dissolution Rate

| Dissolution Time/ | Dissolution Rate (%) | | |
|---|---|---|---|
| n = 12 (average) | Ex. 8 | C. Ex. 7 | C. Ex. 8 |
| 120 min (pH 1.2) | 0.9 ± 0.3% | 3.6 ± 3.1% | 1.5 ± 0.8% |
| 90 min (pH 6.8) | 94.6 ± 0.8% | 94.1 ± 1.5% | 93.7 ± 1.1% |

As is apparent from the above table, the dissolution standards of USP 28 ‘Aspirin Delayed Release Capsule’, that is, 10% or less at pH 1.2 for 120 min and 80% or more at pH 6.8 for 90 min were satisfied in Example 8 and Comparative Examples 7 and 8.

In the acid resistance test, at pH 1.2, the dissolution of Comparative Example 7 was higher than that of Example 8 and the deviation thereof was larger. This is considered to be because the enteric coating is partially damaged due to the friction between adjacent enteric aspirin pellets in the tablet press and the high pressure of the tablet press in the compression procedure upon tableting the mixture of aspirin pellets and pravastatin. Meanwhile, Comparative Example 8 was not significantly different from Example 8.

At pH 6.8, the dissolution rates of Example 8 including the plurality of mini-tablets and of Comparative Examples 7 and 8 including the plurality of mini-pellets were not significantly different.

### (3) Comparison of the Stability of Formulations

The stability of each of Example 8, and Comparative Examples 7 and 8 was tested for 6 months under the following conditions: long storage test conditions: 25° C., 60% RH, acceleration test conditions: 40° C., 75% RH. Respective amounts were measured using Alliance HPLC (available from Water). The results are shown in the Table 39 below (initial amounts of aspirin and pravastatin of Example 8 and Comparative Examples 7 and 8 used in the long storage test and the acceleration test) and the Table 40 below (amounts of aspirin and pravastatin of Example 8 and Comparative Examples 7 and 8 after a 6-month stability test).

TABLE 39
Initial Amounts of Aspirin and Pravastatin

| | Amount (%) | | |
|---|---|---|---|
| Initial | Ex. 8 | C. Ex. 7 | C. Ex. 8 |
| Aspirin | 99.7 | 98.5 | 99.2 |
| Pravastatin | 99.6 | 100.0 | 98.5 |

TABLE 40
Amounts of Aspirin and Pravastatin after 6-Month Stability Test

| | | Amount (%) | | |
|---|---|---|---|---|
| After 6 months | | Ex. 8 | C. Ex. 7 | C. Ex. 8 |
| Long Storage | Aspirin | 98.2 | 95.9 | 97.1 |
| | Pravastatin | 98.2 | 97.2 | 91.5 |
| Acceleration | Aspirin | 96.3 | 92.8 | 96.5 |
| | Pravastatin | 96.1 | 88.9 | 86.2 |

As is apparent from the Table 40, the results of the 6-month stability test were that the amount of the capsules of Example 8 underwent no significant change for 6 months and maintained stability. However, in Comparative Examples 7 and 8, the amounts were remarkably decreased upon 6-month acceleration testing and these formulations were identified as being unstable.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A combined formulation for oral administration for treatment of cardiovascular disease, comprising:
(a) cholesterol lowering agent mini-tablets having a diameter of 7.5 mm or less, which contain a cholesterol lowering agent, a stabilizer thereof and a pharmaceutically acceptable excipient and have a coating layer on a surface thereof; and
(b) antithrombotic agent mini-tablets or mini-pellets having a diameter of 7.5 mm or less, which contain an antithrombotic agent and a pharmaceutically acceptable excipient and include an enteric coating film on a surface thereof.

2. The combined formulation of claim 1, wherein the antithrombotic agent is a salicylic acid derivative which is one or more selected from the group consisting of sodium salicylate, magnesium salicylate (including tetrahydrate), salicylsalicylic acid (salsalate) and aspirin.

3. The combined formulation of claim 2, wherein the salicylic acid derivative is aspirin.

4. The combined formulation of claim 1, wherein the cholesterol lowering agent is one or more pharmaceutically acceptable HMG-CoA (3-hydroxy-3-methylglutaryl-coenzyme A) reductase inhibitors selected from the group consisting of rosuvastatin, rosuvastatin calcium, atorvastatin, atorvastatin calcium, pitavastatin, and pharmaceutically acceptable salts thereof.

5. The combined formulation of claim 1, wherein the stabilizer is one or more selected from the group consisting of butylated hydroxytoluene (BHT), dibutylhydroxytoluene (DHT), butylated hydroxyanisole (BHA), sodium sulfite, sodium pyrosulfite, sodium bisulfate, propyl gallate, and calcium phosphate.

6. The combined formulation of claim 1, wherein the pharmaceutically acceptable excipient included in the cholesterol lowering agent mini-tablet is one or more selected from the group consisting of a diluent, a binder, a disintegrant, and a lubricant.

7. The combined formulation of claim 6, wherein the diluent is microcrystalline cellulose, the stabilizer is calcium phosphate, the binder is one or more selected from the group consisting of anhydrous lactose, povidone and copovidone, the disintegrant is crospovidone, and the lubricant is magnesium stearate.

8. The combined formulation of claim 1, wherein the pharmaceutically acceptable excipient included in the antithrombotic agent mini-tablet or mini-pellet is one or more selected from the group consisting of a diluent and a binder.

9. The combined formulation of claim 8, wherein the diluent is microcrystalline cellulose, and the binder is one or more selected from the group consisting of hydroxypropylcellulose and copovidone.

10. The combined formulation of claim 1, wherein the diameter of the cholesterol lowering agent mini-tablet is 1.5˜7.5 mm.

11. The combined formulation of claim 1, wherein the diameter of the antithrombotic agent mini-tablet is 1.5˜7.5 mm.

12. The combined formulation of claim 1, wherein the mini-tablet is formed via compression using a tablet press equipped with multi-tip punches.

13. The combined formulation of claim 12, wherein the compression is performed at a pressure of 0.5˜1.5 KN.

14. The combined formulation of claim 1, wherein the diameter of the antithrombotic agent mini-pellet is 0.5˜7.5 mm.

15. The combined formulation of claim 1, wherein the enteric coating film is formed from an enteric coating film forming material selected from the group consisting of hydroxypropyl methylcellulose phthalate, methacrylic acid copolymers, cellulose acetate phthalate, ethylcellulose, cellulose acetate, polyvinylacetate and mixtures thereof.

16. The combined formulation of claim 15, wherein an amount of the enteric coating film forming material is 0.01˜0.7 parts by weight based on 1 part by weight of the antithrombotic agent.

17. The combined formulation of claim 1, wherein an amount of the antithrombotic agent in a single dose is 0.5˜500 mg.

18. The combined formulation of claim 1, wherein an amount of the cholesterol lowering agent in a single dose is 1˜300 mg.

19. The combined formulation of claim 1, wherein a dosage form of the combined formulation for oral administration is a capsule.
